(19) United States
(12) Patent Application Publication
(10) Pub. No.: US 2020/0345637 A1
(43) Pub. Date: Nov. 5, 2020

(54) SUSPENSION COMPOSITIONS OF MULTI-TARGET INHIBITORS

(71) Applicant: AIVIVA BIOPHARMA, INC., Las Vegas, NV (US)

(72) Inventors: Diane Tang-Liu, Las Vegas, NV (US); Shulin Ding, Silver Spring, MD (US); Goutam Muhuri, Las Vegas, NV (US)

(21) Appl. No.: 16/961,361

(22) PCT Filed: Jan. 19, 2018
(86) PCT No.: PCT/US2019/014384

§ 371 Date: Jul. 10, 2020

Related U.S. Application Data
(60) Provisional application No. 62/619,354, filed on Jan. 19, 2018.

(51) Int. Cl. A61K 9/08; A61K 47/38; A61K 31/496; A61K 31/47; A61K 31/4439; A61K 31/404; A61K 9/00

(52) U.S. Cl. CPC A61K 9/08; A61K 47/38; A61K 31/496; A61K 9/0019; A61K 31/4439; A61K 31/404; A61K 31/47

(57) ABSTRACT

This disclosure relates to aqueous suspension formulations of multi-target inhibitors. These formulations can be locally administered to target tissues such as eyes, prostate, that may be solved by the present disclosure are to (1) provide an injectable dosage form that allows direct delivery of a multi-target inhibitor to the proximity of the diseased tissue, (2) be compatible with the tissue of the site of administration, (3) form a drug reservoir at the administration site to allow prolonged supply of the drug to the diseased tissue, and thus reduce dosing frequency, (4) the release rate of the drug from the reservoir is such that above therapeutic level of the drug in the diseased tissue is achievable, (5) have physicochemical properties that are suitable for clinical uses, (6) be terminally sterilizable so that safety risk due to contamination of micro-organisms can be minimized, and (7) have a stability profile suitable for long term storage and distribution.

### CROSS-REFERENCE TO RELATED APPLICATIONS

This application claims the benefit of U.S. Provisional Application No. 62/619,354, filed Jan. 19, 2018, which is incorporated by reference herein in its entirety.

### BACKGROUND

### Field

This disclosure relates to aqueous suspension formulations of multi-target inhibitors. These formulations can be locally administered to target tissues such as eyes, prostate, and skin for the treatment of dermatological, ophthalmologic, urogenitary diseases.

### SUMMARY

Some embodiments include compositions (e.g., aqueous suspensions) and methods for treating and/or preventing local diseases/disorders by administering an aqueous suspension to a human or animals. The active ingredients may deposit at the sites of administration and release over time for local actions. The formulations apply to multi-target inhibitors, such as axitinib, nintedanib, pirfenidone, riociguat, sorafenib, sunitinib, lenvatinib, regorafenib, ponatinib, and pazopanib.

Some embodiments include a pharmaceutical composition comprising: an aqueous suspension comprising a pharmaceutically acceptable vehicle and at least one multi-target inhibitor (referred to herein for convenience as a “subject formulation”). Some pharmaceutically acceptable vehicles include a suspending agent, a wetting agent, a buffer system, and/or an osmotic agent.

Some embodiments include a method of treating a dermatological disorder, an ophthalmologic disorder, or an urogenitary disorder, comprising: administering to effective amount a subject formulation to treat a subject suffering from the dermatological disorder, the ophthalmologic disorder, or the urogenitary disorder.

Some embodiments include use of multi-target inhibitors for the manufacture of pharmaceutical compositions for the treatments of dermatological disorders, ophthalmologic disorders, or urogenitary disorders. In some embodiments, the pharmaceutical composition manufactured is a subject formulation.

### BRIEF DESCRIPTION OF THE FIGURES

FIG. 1 depicts the reduction of neovascular lesions in rat choroidal neovascularization model, with VH=vehicle control, NTDN=nintedanib, and LVTN=lenvatinib.

FIG. 2 depicts the plasma concentration of nintedanib and its metabolite and lenvatinib in ng/mL (mean+/−SEM, n=6).

FIG. 3A depicts the concentration of nintedanib and its metabolite in ocular tissue, in ng/mL or ng/gm, after one single intravitreal injection of nintedanib in both eyes on day 2 (IVT).

FIG. 3B depicts the concentration of nintedanib and its metabolite in ocular tissue, in ng/mL or ng/gm, after one 10 μl eyedrop of 1% nintedanib three times daily on day 3-21.

FIG. 3C depicts the concentration of lenvatinib in ocular tissue, in ng/mL or ng/gm, after one single intravitreal injection of lenvatinib in both eyes on day 2 (IVT).

FIG. 3D depicts the concentration of lenvatinib and its metabolite in ocular tissue, in ng/mL or ng/gm, after one 10 μl eyedrop of lenvatinib three times daily on day 3-21.

FIG. 4 depicts representative images of rabbit eyes after a single intravitreal injection of nintedanib suspension.

FIG. 5 depicts representative images of rabbit eyes after a single intravitreal injection of lenvatinib suspension.

FIG. 6 depicts the mean concentration of axitinib (ng/gm) in ocular tissues of rabbits.

FIG. 7 depicts the mean concentration of nintedanib and its metabolite (ng/gm) in ocular tissues of rabbits.

FIG. 8 depicts the mean concentration of lenvatinib (ng/gm) in ocular tissues of rabbits.

FIG. 9 depicts the mean concentration of axitinib (ng/gm) in ocular tissues of rabbits.

FIG. 10 depicts the mean concentration of sorafenib (ng/gm) in ocular tissues of rabbits.

FIG. 11 depicts the mean concentration of lenvatinib (ng/gm) in ocular tissues of rabbits.

FIG. 12 depicts the plasma concentration of nintedanib and its metabolite (ng/mL) after intraprostate injections in rats.

FIG. 13 depicts the plasma concentration of lenvatinib (ng/mL) after intraprostate injections in rat.

FIG. 14 depicts the concentration of nintedanib and its metabolite (μg/gm) in the dorsolateral and ventral lobes after intraprostate injections in rats.

FIG. 15 depicts the concentration of lenvatinib (μg/gm) in the dorsolateral and ventral lobes after intraprostate injections in rats.

FIG. 16. depicts a summary of histological evaluation of fibroplasia (dermis and subcutis), dermis fibrin, and alpha sma staining in wounds treated with axitinib, nintedanib, sorafenib, lenvatinib, and vehicle.

### DETAILED DESCRIPTION

Targeted drug delivery to local tissues may be preferred over systemic drug administration. It may be employed to improve the local drug exposure and reduce systemic side effects. There are some considerations with respect to local drug delivery that may improve the result. For example, it may be helpful if the method of drug delivery to the local tissue is easily administered by the patients or by the medical practitioners with adequate local tolerability and safety. It may also be useful if the dose and dose volume are appropriate for the volume of the target tissue and provide effective disease management. It may also be desirable for the drug to be released over a suitable period of time.

Surprisingly, some subject formulations provide sufficient amounts of active agents to target tissues through local applications and are effective for treating dermatological, ophthalmologic and urogenitary disorders

Some subject formulations include suspension compositions of multi-target inhibitors with certain water solubilities, e.g. approximately 10 mg/ml and below.

Low solubility multi-target inhibitors may be challenging to formulate into pharmaceutically acceptable injectable suspensions. Some of the reasons are as follows:
1. Suspensions are a dispersed system, which is inherently thermodynamically unstable. Pharmaceutical suspensions are usually solids dispersed in a liquid. The solid particles may settle and cake, causing difficulty in re-dispersion prior to use. The solids may undergo polymorphic transformation during storage if the polymorphic form formulated is not the most thermodynamically stable form. Furthermore, the particles may grow in size due to Ostwald ripening phenomenon. The phenomenon could result in a significant shift in particle size distribution and alter the bioavailability of the product through an alteration in the dissolution rate. Thus, it can be challenging to achieve a suspension with acceptable physical stability.
2. The pharmaceutically acceptable excipients for parenteral suspensions are limited. In order to reduce the thermodynamic instability, it may be desirable to incorporate suspending agents, wetting agents, viscosity enhancers, surfactants, flocculation agents, etc. Unfortunately, many of these functional excipients have not had an adequate history for pharmaceutical parenteral uses.
3. Highly specialized facility, equipment, and manufacturing processes may be needed for the manufacture of parenteral suspensions in order to produce sterile products that meet commercial and regulatory standards.

The subject formulations are biocompatible with tissues of human and animals. Some formulations may be chemically and physically stable, and may be resuspendable upon storage to regain uniformity. Some formulations can undergo terminal sterilization without losing their physicochemical integrities. Some formulations have low endotoxin as well as particulate matter contamination. Some subject formulations can be injected into local tissues using needle gauge sizes typically used medically. Some formulations described herein have demonstrated acceptable stability profiles and are amendable for long-term storage and distributions.

Some embodiments relate to a pharmaceutical aqueous suspension formulation composition, which may comprise a pharmaceutically acceptable vehicle and at least one multi-target inhibitor.

Unless otherwise indicated, any reference to a compound herein, such as a multi-target inhibitor, by structure, name, or any other means, includes free bases, free acids, pharmaceutically acceptable salts, alternate solid forms (such as polymorphs, solvates, hydrates, etc.) enantiomers, tautomers, prodrugs, or any other chemical species that may rapidly convert to a compound described herein under conditions in which the compounds are used as described herein.

The physical form of the active ingredients in these suspensions described herein may be a solid, which can be amorphous or a polymorph. A solid active ingredient may have any suitable particle size, such as between about 0.1-100 μm, about 1-20 μm, or about 1-10 μm.

Non-limiting examples of the useful multi-target inhibitors and their physicochemical forms are given in Table 1 with aqueous solubility information.

TABLE 1
Examples of Multi-Target Inhibitors: Structures, Physicochemical Forms, and
Aqueous Solubilities

| Multi- | | Physical | Molecular | | |
|---|---|---|---|---|---|
| Target | Chemical | Form and | Weight | Aqueous | |
| Inhibitors | Form | Polymorph | (g/mole) | Solubility | Structure |
| Axitinib | Free base | Solid, Form IV | 386 | 2 μg/mL | 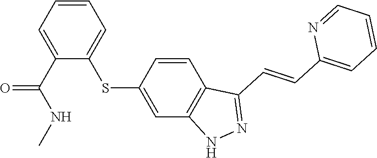 [SMILES: CNC(=O)c1ccccc1Sc1ccc2c(/C=C/c3ccccn3)n[nH]c2c1] |
| Nintedanib | Free base | Solid | 539 | 9 μg/mL | 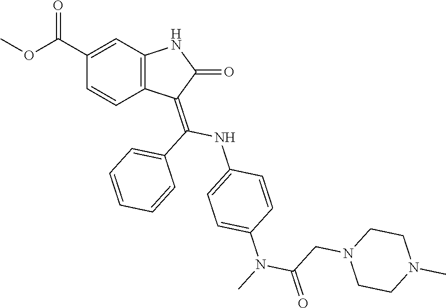 |
| Pirfenidone | Pyridone | Solid | 185 | 3 mg/mL | 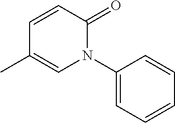 |
| Riociguat | Free base | Solid | 422 | 8 μg/mL | 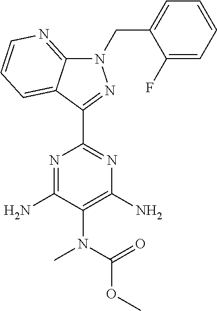 [SMILES: COC(=O)N(C)c1c(N)nc(-c2nn(Cc3ccccc3F)c3ncccc23)nc1N] |
| Sorafenib Hemi Tosylate | Salt | Solid, Form A | 636 | 6 μg/mL | 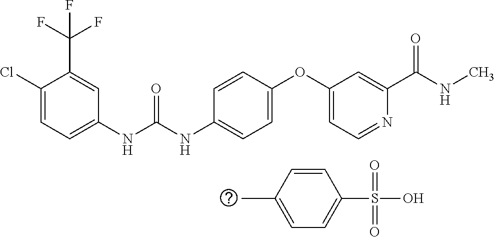 |
| Sunitinib | Free base | Solid, Form-I | 398 | 7 μg/mL | 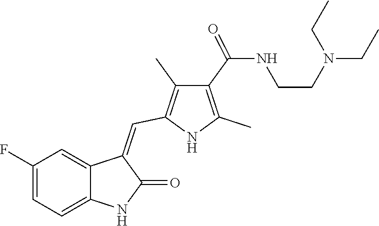 [SMILES: CCN(CC)CCNC(=O)c1c(C)[nH]c(/C=C2\C(=O)Nc3ccc(F)cc32)c1C] |
| Lenvatinib | Free base | Solid, Form B | 426 | 4 μg/mL | 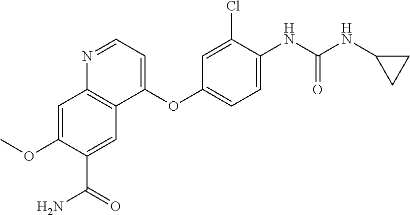 [SMILES: COc1cc2nccc(Oc3ccc(NC(=O)NC4CC4)c(Cl)c3)c2cc1C(N)=O] |
| Regorafenib | Free base | Solid | 482 | | 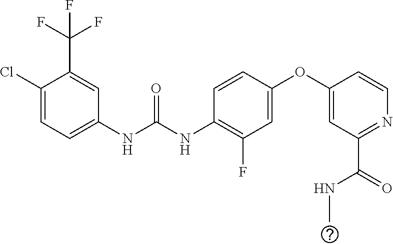 [SMILES: CNC(=O)c1cc(Oc2ccc(NC(=O)Nc3ccc(Cl)c(C(F)(F)F)c3)c(F)c2)ccn1] |
| Ponatinib | Free base | Solid | 532 | | 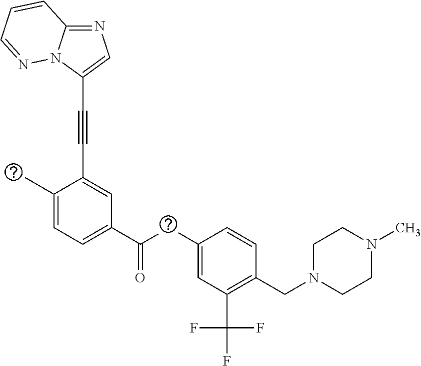 [SMILES: Cc1ccc(C(=O)Cc2ccc(CN3CCN(C)CC3)c(C(F)(F)F)c2)cc1C#Cc1cnc2cccnn12] |
| Pazopanib | Free base | Solid | 437 | | 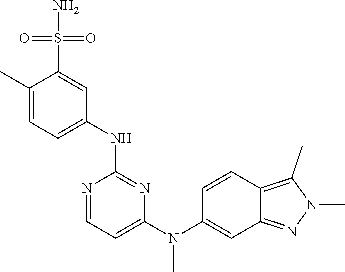 |

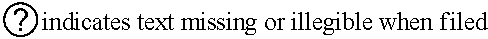

The pharmaceutically acceptable vehicle may comprise at least one suitable suspending agent, at least one suitable surfactant as wetting agent, at least one suitable buffer system, and/or at least one suitable osmotic agent. The suspending agent may be sodium carboxymethylcellulose, cellulose, or a mixture of multiple celluloses. The wetting agent may be a pharmaceutically acceptable non-ionic surfactant, such as polysorbate 80. The buffer system may be a pharmaceutically acceptable buffering system for parenteral formulations to control the pH values near physiological pH. The buffer system may be a phosphate buffer. The osmotic agents may comprise sodium chloride and/or glycerin.

Some subject formulation may further comprise a suitable preservative, such as benzyl alcohol. Some embodiments may further comprise additional ingredients, such as a viscosity enhancer, a stabilizer, a chelating agent, an anti-oxidant, an organic, or a co-solvent.

The multi-target inhibitor may be present in any suitable concentration or amount in a subject formulation, such as about 0.01-20%, 0.01-10%, about 0.01-8%, about 0.1-4%, about 4-8%, about 0.01-3%, about 0.01-2%, about 2-4%, about 0.01-0.5%, about 0.5-1%, about 1-1.5%, about 1.5-2%, about 0.01-1%, about 1-2%, about 2-3%, about 3-4%, about 4-5%, about 5-6%, about 6-7%, about 7-8%, about 8-9%, about 9-10%, about 0.01-3%, about 3-5%, about 5-10%, by weight of the total amount of the composition.

In some embodiments, the multi-target inhibitor in the subject formulation is axitinib, which is present in a concentration or amount of about 0.01-10%, 0.01-8%, about 0.1-4%, about 4-8%, about 0.01-3%, about 0.01-2%, about 2-4%, about 0.01-0.5%, about 0.5-1%, about 1-1.5%, about 1.5-2%, about 0.01-1%, about 1-2%, about 2-3%, about 3-4%, about 4-5%, about 5-6%, about 6-7%, or about 7-8%, by weight of the total amount of the composition.

In some embodiments, the multi-target inhibitor in the subject formulation is nintedanib, which is present in a concentration or amount of about 0.01-10%, 0.01-8%, about 0.1-4%, about 4-8%, about 0.01-3%, about 0.01-2%, about 2-4%, about 0.01-0.5%, about 0.5-1%, about 1-1.5%, about 1.5-2%, about 0.01-1%, about 1-2%, about 2-3%, about 3-4%, about 4-5%, about 5-6%, about 6-7%, or about 7-8%, by weight of the total amount of the composition.

In some embodiments, the multi-target inhibitor in the subject formulation is pirfenidone, which is present in a concentration or amount of about 0.01-10%, 0.01-8%, about 0.1-4%, about 4-8%, about 0.01-3%, about 0.01-2%, about 2-4%, about 0.01-0.5%, about 0.5-1%, about 1-1.5%, about 1.5-2%, about 0.01-1%, about 1-2%, about 2-3%, about 3-4%, about 4-5%, about 5-6%, about 6-7%, or about 7-8%, by weight of the total amount of the composition.

In some embodiments, the multi-target inhibitor in the subject formulation is riociguat, which is present in a concentration or amount of about 0.01-10%, 0.01-8%, about 0.1-4%, about 4-8%, about 0.01-3%, about 0.01-2%, about 2-4%, about 0.01-0.5%, about 0.5-1%, about 1-1.5%, about 1.5-2%, about 0.01-1%, about 1-2%, about 2-3%, about 3-4%, about 4-5%, about 5-6%, about 6-7%, or about 7-8%, by weight of the total amount of the composition.

In some embodiments, the multi-target inhibitor in the subject formulation is sorafenib, which is present in a concentration or amount of about 0.01-10%, 0.01-8%, about 0.1-4%, about 4-8%, about 0.01-3%, about 0.01-2%, about 2-4%, about 0.01-0.5%, about 0.5-1%, about 1-1.5%, about 1.5-2%, about 0.01-1%, about 1-2%, about 2-3%, about 3-4%, about 4-5%, about 5-6%, about 6-7%, or about 7-8%, by weight of the total amount of the composition.

In some embodiments, the multi-target inhibitor in the subject formulation is sunitinib, which is present in a concentration or amount of about 0.01-10%, 0.01-8%, about 0.1-4%, about 4-8%, about 0.01-3%, about 0.01-2%, about 2-4%, about 0.01-0.5%, about 0.5-1%, about 1-1.5%, about 1.5-2%, about 0.01-1%, about 1-2%, about 2-3%, about 3-4%, about 4-5%, about 5-6%, about 6-7%, or about 7-8%, by weight of the total amount of the composition.

In some embodiments, the multi-target inhibitor in the subject formulation is lenvatinib, which is present in a concentration or amount of about 0.01-10%, 0.01-8%, about 0.1-4%, about 4-8%, about 0.01-3%, about 0.01-2%, about 2-4%, about 0.01-0.5%, about 0.5-1%, about 1-1.5%, about 1.5-2%, about 0.01-1%, about 1-2%, about 2-3%, about 3-4%, about 4-5%, about 5-6%, about 6-7%, or about 7-8%, by weight of the total amount of the composition.

In some embodiments, the multi-target inhibitor in the subject formulation is regorafenib, which is present in a concentration or amount of about 0.01-10%, 0.01-8%, about 0.1-4%, about 4-8%, about 0.01-3%, about 0.01-2%, about 2-4%, about 0.01-0.5%, about 0.5-1%, about 1-1.5%, about 1.5-2%, about 0.01-1%, about 1-2%, about 2-3%, about 3-4%, about 4-5%, about 5-6%, about 6-7%, or about 7-8%, by weight of the total amount of the composition.

In some embodiments, the multi-target inhibitor in the subject formulation is ponatinib, which is present in a concentration or amount of about 0.01-10%, 0.01-8%, about 0.1-4%, about 4-8%, about 0.01-3%, about 0.01-2%, about 2-4%, about 0.01-0.5%, about 0.5-1%, about 1-1.5%, about 1.5-2%, about 0.01-1%, about 1-2%, about 2-3%, about 3-4%, about 4-5%, about 5-6%, about 6-7%, or about 7-8%, by weight of the total amount of the composition.

In some embodiments, the multi-target inhibitor in the subject formulation is pazopanib, which is present in a concentration or amount of about 0.01-10%, 0.01-8%, about 0.1-4%, about 4-8%, about 0.01-3%, about 0.01-2%, about 2-4%, about 0.01-0.5%, about 0.5-1%, about 1-1.5%, about 1.5-2%, about 0.01-1%, about 1-2%, about 2-3%, about 3-4%, about 4-5%, about 5-6%, about 6-7%, or about 7-8%, by weight of the total amount of the composition.

A multi-target inhibitor (such as axitinib, nintedanib, pirfenidone, riociguat, sorafenib, sunitinib, lenvatinib, regorafenib, ponatinib, or pazopanib) may be administered by injection (such as in an amount in the preceding paragraphs) as needed, or at an interval of approximately weekly to approximately every 2 years, such as at an interval of about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 27 weeks, about 28 weeks, about 29 weeks, about 30 weeks, about 31 weeks, about 32 weeks, about 33 weeks, about 34 weeks, about 35 weeks, about 36 weeks, about 37 weeks, about 38 weeks, about 39 weeks, about 40 weeks, about 41 weeks, about 42 weeks, about 43 weeks, about 44 weeks, about 45 weeks, about 46 weeks, about 47 weeks, about 48 weeks, about 49 weeks, about 50 weeks, about 51 weeks, about 52 weeks, about 1 month, about 2 months, about 3 months, about 4 months, about 5 months, about 6 months, about 7 months, about 8 months, about 9 months, about 10 months, about 11 months, about 12 months, about 18 months, about 24 months, etc.

The treatment in the preceding paragraphs (e.g. a multi-target inhibitor such as axitinib, nintedanib, pirfenidone, riociguat, sorafenib, sunitinib, lenvatinib, regorafenib, ponatinib, or pazopanib, administered in amounts and intervals described above) may be continued for as long as needed, such as only once, or for at least about 2 weeks, at least about 3 weeks, at least about 4 weeks, at least about 5 weeks, at least about 2 months, at least about 3 months, at least about 6 months, at least about 9 months, at least about 12 months, at least about 2 years, at least about 3 years, at least about 4 years, at least about 5 years, at least about 10 years, or at least about 20 years.

A subject formulation may optionally contain a suspending agent, or an agent added to help in suspending the particles, such as the solid particles, into the aqueous solvent. A suspending agent may a polymer, including a biopolymer or a derivative thereof, or a mineral. Examples of suitable suspending agents include an alginate, a methylcellulose, a hydroxyethylcellulose, a carboxymethylcellulose, a sodium carboxymethylcellulose, a microcrystalline cellulose, an acacia gum, a tragacanth, a xanthan gum, a bentonite, a carbomer, a carageenan, a powdered cellulose, a gelatin, etc.

A suspending agent (e.g. a carboxymethylcellulose such as sodium carboxymethylcellulose, cellulose, a mixture of celluloses, etc.) may be present in any suitable concentration or amount in a subject formulation, such as about 0.1-10%, about 0.2-3%, about 3-6%, about 6-10%, about 0.1-1%, about 1-2%, about 2-3%, about 3-4%, about 4-5%, about 5-6%, about 6-7%, about 7-8%, about 8-9%, about 9-10%, or by weight of total composition. In some embodiments, the suspending agent is a carboxymethylcellulose, such as sodium carboxymethylcellulose. In some embodiments, the suspending agent is a cellulose. In some embodiments, the suspending agent is a mixture of multiple celluloses.

A subject formulation may optionally contain a wetting agent to help improve wetting of a low solubility multi-target inhibitor by the aqueous solution. Suitable wetting agents my include co-solvents, hydrotropes, surfactants, or the like. In some embodiments, the wetting agent is a pharmaceutically acceptable surfactant, such as a nonionic surfactant, e.g. an alkylene oxide based surfactant (such as an ethylene oxide-propylene oxide block copolymer, a fatty acid polyethylene oxide, a sugar based polyethylene oxide such as a polysorbate, etc.), an amphoteric surfactant, such as an amine oxide or a betaine, an anionic surfactant, such as an alkyl sulfate, an alkylbenzenesulfonate, an alkylether sulfate etc., or a cationic surfactant, such as a quaternary amine compound. In some embodiments, the wetting agent is a pharmaceutically acceptable non-ionic surfactant. In some embodiments, the wetting agent is a polysorbate, such a polysorbate 80.

A subject formulation may optionally contain any suitable amount of surfactant (e.g. polysorbate 80), such as about 0.02-5%, about 0.02-3%, about 3-5%, about 0.2-1%, about 1-2%, about 2-3%, about 0.2-1%, about 1-1.5%, about 1.5-2%, about 2-2.5%, or about 2.5-3% of the total weight of the subject formulation.

A subject formulation may optionally contain a buffer system. Suitable buffers include citrate, phosphate, carbonate, bicarbonate, borate, etc. Any suitable concentration of buffer (e.g. phosphate buffer) may be used, such as about 0.01-5%, about 0.1-2%, about 0.1-0.2%, about 0.2-0.4%, about 0.4-0.6%, about 0.6-0.8%, about 0.8-1%, about 0.01-0.5%, about 0.5-1%, about 0.01-1%, about 1-2%, about 2-3%, about 3-4%, or about 4-5% of the total weight of the subject formulation.

A subject formulation may have any suitable pH, such as a pH near or around the pH of the tissue to which it is delivered e.g. about 5-9, about 6-8, about 5-7, about 7-9, about 5-6, about 6-7, about 7-8, about 8-9, about 7-7.2, about 7.2-7.4, about 7.4-7.6, about 7.6-7.8, about 7.8-8, etc.

A subject formulation may optionally contain an osmotic agent, such as dextrose, glycerin, mannitol, sodium chloride, etc. Any suitable amount of osmotic agent (such as sodium chloride or glycerine) may be used, such as about 0.01-2%, about 0.1-1%, about 0.1-0.5%, about 0.5-1%, about 0.01-0.2%, about 0.2-0.4%, about 0.4-0.6%, about 0.6-0.8%, about 0.8-1%, about 0.5%, or about 0.75% of the total weight of the subject formulation.

A subject formulation may optionally contain a preservative, such as phenol, m-cresol, a paraben, such as methylparaben, propylparaben, butylparaben, myristyl gamma-picolinium chloride, benzalkonium chloride, benzethonium chloride, benzyl alcohol, 2-penoxyethanol, chlorobutanol, thimerosal, phenymercuric salts, etc. Any suitable amount of preservative (such as benzyl alcohol) may be used, such as about 0.01-2%, about 0.5-2%, about 0.01-0.2%, about 0.2-0.4%, about 0.4-0.6%, about 0.6-0.8%, about 0.8-1%, about 1-1.2%, about 1.2-1.4%, about 1.4-1.6%, about 1.6-1.8%, about 1.8-2%, about 0.01-0.5%, about 0.5-1%, about 1-1.5%, about 1.5-2%, or about 0.9% of the total weight of the subject formulation.

A subject formulation may have the property that, when injected into a prostate of mammal such as a rabbit or a human being, the concentration of the multi-target inhibitor (such as axitinib, nintedanib, pirfenidone, riociguat, sorafenib, sunitinib, lenvatinib, regorafenib, ponatinib, or pazopanib) in prostate tissue, is at least about 0.1 μg/g, at least 0.5 μg/g, at least about 1 μg/g, at least about 5 μg/g, or at least about 10 μg/g after about 7 days, 10 days, 15 days, 20 days, 30 days, 40 days, 50 days, 60 days or more.

A subject formulation may have the property that, when injected intraocularly into a a mammal such as a rabbit or a human being, the concentration of the multi-target inhibitor (such as axitinib, nintedanib, pirfenidone, riociguat, sorafenib, sunitinib, lenvatinib, regorafenib, ponatinib, or pazopanib) in posterior ocular tissue, such as the vitreous humor, the retina, the choroid, etc. is at least about 0.1 μg/g, at least 0.5 μg/g, at least about 1 μg/g, at least about 5 μg/g, or at least about 10 μg/g after about 7 days, 10 days, 15 days, 20 days, 30 days, 40 days, 50 days, 60 days or more.

In some embodiments, the dermatological disorder includes, but is not limited to, acne scar, skin scar, wrinkle, cellulite and dermal neoplastic fibrosis, scarring alopecia, vasculopathy, vasculitis, wound healing, exuberant burn wound healing, diabetic foot syndrome, scleroderma, arthrofibrosis, Peyronie's disease, Dupuytren's contracture and adhesive capsulitis.

In some embodiments, the ophthalmologic disorder includes, but is not limited to, choroidal neovascularization associated diseases, age-related macular degeneration, compromised corneal transparency, cornea scar formation, pterygia anterior cataract formation, a condition related to glaucoma filtration surgery, glaucoma, a condition related to photorefractive keratectomy, a condition related to laser in situ keratomileusis, disorders related contraction of the pre- and epiretinal membranes, proliferative vitreoretinopathy, proliferative diabetic retinopathy, diabetic macular edema, disorders related to myopic choroidal neovascularization, retinal vein occlusion, sub-retinal fibrosis, sub-retinal scarring, choroidal membranes related disorders, retinal gliosis, epiretinal membranes related disorders and glial scar formation.

In some embodiments, the urogenitary disorder includes, but is not limited to, benign prostate hyperplasia, lower urinary tract symptoms, benign prostatic enlargement, bladder outlet obstruction, overactive bladder-related disorders, prostatitis, prostatic intraepithelial neoplasia, neurogenic bladder syndrome, prostate cancer, urinary incontinence, prostate cancer and pelvic pain.

In some embodiments, the subject formulation comprises axitinib, and the subject formulation is used to treat acne scar.

In some embodiments, the subject formulation comprises nintedanib, and the subject formulation is used to treat acne scar.

In some embodiments, the subject formulation comprises pirfenidone, and the subject formulation is used to treat acne scar.

In some embodiments, the subject formulation comprises riociguat, and the subject formulation is used to treat acne scar.

In some embodiments, the subject formulation comprises sorafenib, and the subject formulation is used to treat acne scar.

In some embodiments, the subject formulation comprises sunitinib, and the subject formulation is used to treat acne scar.

In some embodiments, the subject formulation comprises lenvatinib, and the subject formulation is used to treat acne scar.

In some embodiments, the subject formulation comprises regorafenib, and the subject formulation is used to treat acne scar.

In some embodiments, the subject formulation comprises ponatinib, and the subject formulation is used to treat acne scar.

In some embodiments, the subject formulation comprises pazopanib, and the subject formulation is used to treat acne scar.

In some embodiments, the subject formulation comprises axitinib, and the subject formulation is used to treat skin scar.

In some embodiments, the subject formulation comprises nintedanib, and the subject formulation is used to treat skin scar.

In some embodiments, the subject formulation comprises pirfenidone, and the subject formulation is used to treat skin scar.

In some embodiments, the subject formulation comprises riociguat, and the subject formulation is used to treat skin scar.

In some embodiments, the subject formulation comprises sorafenib, and the subject formulation is used to treat skin scar.

In some embodiments, the subject formulation comprises sunitinib, and the subject formulation is used to treat skin scar.

In some embodiments, the subject formulation comprises lenvatinib, and the subject formulation is used to treat skin scar.

In some embodiments, the subject formulation comprises regorafenib, and the subject formulation is used to treat skin scar.

In some embodiments, the subject formulation comprises ponatinib, and the subject formulation is used to treat skin scar.

In some embodiments, the subject formulation comprises pazopanib, and the subject formulation is used to treat skin scar.

In some embodiments, the subject formulation comprises axitinib, and the subject formulation is used to treat wrinkle.

In some embodiments, the subject formulation comprises nintedanib, and the subject formulation is used to treat wrinkle.

In some embodiments, the subject formulation comprises pirfenidone, and the subject formulation is used to treat wrinkle.

In some embodiments, the subject formulation comprises riociguat, and the subject formulation is used to treat wrinkle.

In some embodiments, the subject formulation comprises sorafenib, and the subject formulation is used to treat wrinkle.

In some embodiments, the subject formulation comprises sunitinib, and the subject formulation is used to treat wrinkle.

In some embodiments, the subject formulation comprises lenvatinib, and the subject formulation is used to treat wrinkle.

In some embodiments, the subject formulation comprises regorafenib, and the subject formulation is used to treat wrinkle.

In some embodiments, the subject formulation comprises ponatinib, and the subject formulation is used to treat wrinkle.

In some embodiments, the subject formulation comprises pazopanib, and the subject formulation is used to treat wrinkle.

In some embodiments, the subject formulation comprises axitinib, and the subject formulation is used to treat cellulite.

In some embodiments, the subject formulation comprises nintedanib, and the subject formulation is used to treat cellulite.

In some embodiments, the subject formulation comprises pirfenidone, and the subject formulation is used to treat cellulite.

In some embodiments, the subject formulation comprises riociguat, and the subject formulation is used to treat cellulite.

In some embodiments, the subject formulation comprises sorafenib, and the subject formulation is used to treat cellulite.

In some embodiments, the subject formulation comprises sunitinib, and the subject formulation is used to treat cellulite.

In some embodiments, the subject formulation comprises lenvatinib, and the subject formulation is used to treat cellulite.

In some embodiments, the subject formulation comprises regorafenib, and the subject formulation is used to treat cellulite.

In some embodiments, the subject formulation comprises ponatinib, and the subject formulation is used to treat cellulite.

In some embodiments, the subject formulation comprises pazopanib, and the subject formulation is used to treat cellulite.

In some embodiments, the subject formulation comprises axitinib, and the subject formulation is used to treat dermal neoplastic fibrosis.

In some embodiments, the subject formulation comprises nintedanib, and the subject formulation is used to treat dermal neoplastic fibrosis.

In some embodiments, the subject formulation comprises pirfenidone, and the subject formulation is used to treat dermal neoplastic fibrosis.

In some embodiments, the subject formulation comprises riociguat, and the subject formulation is used to treat dermal neoplastic fibrosis.

In some embodiments, the subject formulation comprises sorafenib, and the subject formulation is used to treat dermal neoplastic fibrosis.

In some embodiments, the subject formulation comprises sunitinib, and the subject formulation is used to treat dermal neoplastic fibrosis.

In some embodiments, the subject formulation comprises lenvatinib, and the subject formulation is used to treat dermal neoplastic fibrosis.

In some embodiments, the subject formulation comprises regorafenib, and the subject formulation is used to treat dermal neoplastic fibrosis.

In some embodiments, the subject formulation comprises ponatinib, and the subject formulation is used to treat dermal neoplastic fibrosis.

In some embodiments, the subject formulation comprises pazopanib, and the subject formulation is used to treat dermal neoplastic fibrosis.

In some embodiments, the subject formulation comprises axitinib, and the subject formulation is used to treat scarring alopecia.

In some embodiments, the subject formulation comprises nintedanib, and the subject formulation is used to treat scarring alopecia.

In some embodiments, the subject formulation comprises pirfenidone, and the subject formulation is used to treat scarring alopecia.

In some embodiments, the subject formulation comprises riociguat, and the subject formulation is used to treat scarring alopecia.

In some embodiments, the subject formulation comprises sorafenib, and the subject formulation is used to treat scarring alopecia.

In some embodiments, the subject formulation comprises sunitinib, and the subject formulation is used to treat scarring alopecia.

In some embodiments, the subject formulation comprises lenvatinib, and the subject formulation is used to treat scarring alopecia.

In some embodiments, the subject formulation comprises regorafenib, and the subject formulation is used to treat scarring alopecia.

In some embodiments, the subject formulation comprises ponatinib, and the subject formulation is used to treat scarring alopecia.

In some embodiments, the subject formulation comprises pazopanib, and the subject formulation is used to treat scarring alopecia.

In some embodiments, the subject formulation comprises axitinib, and the subject formulation is used to treat vasculopathy.

In some embodiments, the subject formulation comprises nintedanib, and the subject formulation is used to treat vasculopathy.

In some embodiments, the subject formulation comprises pirfenidone, and the subject formulation is used to treat vasculopathy.

In some embodiments, the subject formulation comprises riociguat, and the subject formulation is used to treat vasculopathy.

In some embodiments, the subject formulation comprises sorafenib, and the subject formulation is used to treat vasculopathy.

In some embodiments, the subject formulation comprises sunitinib, and the subject formulation is used to treat vasculopathy.

In some embodiments, the subject formulation comprises lenvatinib, and the subject formulation is used to treat vasculopathy.

In some embodiments, the subject formulation comprises regorafenib, and the subject formulation is used to treat vasculopathy.

In some embodiments, the subject formulation comprises ponatinib, and the subject formulation is used to treat vasculopathy.

In some embodiments, the subject formulation comprises pazopanib, and the subject formulation is used to treat vasculopathy.

In some embodiments, the subject formulation comprises axitinib, and the subject formulation is used to treat vasculitis.

In some embodiments, the subject formulation comprises nintedanib, and the subject formulation is used to treat vasculitis.

In some embodiments, the subject formulation comprises pirfenidone, and the subject formulation is used to treat vasculitis.

In some embodiments, the subject formulation comprises riociguat, and the subject formulation is used to treat vasculitis.

In some embodiments, the subject formulation comprises sorafenib, and the subject formulation is used to treat vasculitis.

In some embodiments, the subject formulation comprises sunitinib, and the subject formulation is used to treat vasculitis.

In some embodiments, the subject formulation comprises lenvatinib, and the subject formulation is used to treat vasculitis.

In some embodiments, the subject formulation comprises regorafenib, and the subject formulation is used to treat vasculitis.

In some embodiments, the subject formulation comprises ponatinib, and the subject formulation is used to treat vasculitis.

In some embodiments, the subject formulation comprises pazopanib, and the subject formulation is used to treat vasculitis.

In some embodiments, the subject formulation comprises axitinib, and the subject formulation is used to treat a wound.

In some embodiments, the subject formulation comprises nintedanib, and the subject formulation is used to treat a wound.

In some embodiments, the subject formulation comprises pirfenidone, and the subject formulation is used to treat a wound.

In some embodiments, the subject formulation comprises riociguat, and the subject formulation is used to treat a wound.

In some embodiments, the subject formulation comprises sorafenib, and the subject formulation is used to treat a wound.

In some embodiments, the subject formulation comprises sunitinib, and the subject formulation is used to treat a wound.

In some embodiments, the subject formulation comprises lenvatinib, and the subject formulation is used to treat a wound.

In some embodiments, the subject formulation comprises regorafenib, and the subject formulation is used to treat a wound.

In some embodiments, the subject formulation comprises ponatinib, and the subject formulation is used to treat a wound.

In some embodiments, the subject formulation comprises pazopanib, and the subject formulation is used to treat a wound.

In some embodiments, the subject formulation comprises axitinib, and the subject formulation is used to treat an exuberant burn wound.

In some embodiments, the subject formulation comprises nintedanib, and the subject formulation is used to treat an exuberant burn wound.

In some embodiments, the subject formulation comprises pirfenidone, and the subject formulation is used to treat an exuberant burn wound.

In some embodiments, the subject formulation comprises riociguat, and the subject formulation is used to treat an exuberant burn wound.

In some embodiments, the subject formulation comprises sorafenib, and the subject formulation is used to treat an exuberant burn wound.

In some embodiments, the subject formulation comprises sunitinib, and the subject formulation is used to treat an exuberant burn wound.

In some embodiments, the subject formulation comprises lenvatinib, and the subject formulation is used to treat an exuberant burn wound.

In some embodiments, the subject formulation comprises regorafenib, and the subject formulation is used to treat an exuberant burn wound.

In some embodiments, the subject formulation comprises ponatinib, and the subject formulation is used to treat an exuberant burn wound.

In some embodiments, the subject formulation comprises pazopanib, and the subject formulation is used to treat an exuberant burn wound.

In some embodiments, the subject formulation comprises axitinib, and the subject formulation is used to treat diabetic foot syndrome.

In some embodiments, the subject formulation comprises nintedanib, and the subject formulation is used to treat diabetic foot syndrome.

In some embodiments, the subject formulation comprises pirfenidone, and the subject formulation is used to treat diabetic foot syndrome.

In some embodiments, the subject formulation comprises riociguat, and the subject formulation is used to treat diabetic foot syndrome.

In some embodiments, the subject formulation comprises sorafenib, and the subject formulation is used to treat diabetic foot syndrome.

In some embodiments, the subject formulation comprises sunitinib, and the subject formulation is used to treat diabetic foot syndrome.

In some embodiments, the subject formulation comprises lenvatinib, and the subject formulation is used to treat diabetic foot syndrome.

In some embodiments, the subject formulation comprises regorafenib, and the subject formulation is used to treat diabetic foot syndrome.

In some embodiments, the subject formulation comprises ponatinib, and the subject formulation is used to treat diabetic foot syndrome.

In some embodiments, the subject formulation comprises pazopanib, and the subject formulation is used to treat diabetic foot syndrome.

In some embodiments, the subject formulation comprises axitinib, and the subject formulation is used to treat scleroderma.

In some embodiments, the subject formulation comprises nintedanib, and the subject formulation is used to treat scleroderma.

In some embodiments, the subject formulation comprises pirfenidone, and the subject formulation is used to treat scleroderma.

In some embodiments, the subject formulation comprises riociguat, and the subject formulation is used to treat scleroderma.

In some embodiments, the subject formulation comprises sorafenib, and the subject formulation is used to treat scleroderma.

In some embodiments, the subject formulation comprises sunitinib, and the subject formulation is used to treat scleroderma.

In some embodiments, the subject formulation comprises lenvatinib, and the subject formulation is used to treat scleroderma.

In some embodiments, the subject formulation comprises regorafenib, and the subject formulation is used to treat scleroderma.

In some embodiments, the subject formulation comprises ponatinib, and the subject formulation is used to treat scleroderma.

In some embodiments, the subject formulation comprises pazopanib, and the subject formulation is used to treat scleroderma.

In some embodiments, the subject formulation comprises axitinib, and the subject formulation is used to treat arthrofibrosis.

In some embodiments, the subject formulation comprises nintedanib, and the subject formulation is used to treat arthrofibrosis.

In some embodiments, the subject formulation comprises pirfenidone, and the subject formulation is used to treat arthrofibrosis.

In some embodiments, the subject formulation comprises riociguat, and the subject formulation is used to treat arthrofibrosis.

In some embodiments, the subject formulation comprises sorafenib, and the subject formulation is used to treat arthrofibrosis.

In some embodiments, the subject formulation comprises sunitinib, and the subject formulation is used to treat arthrofibrosis.

In some embodiments, the subject formulation comprises lenvatinib, and the subject formulation is used to treat arthrofibrosis.

In some embodiments, the subject formulation comprises regorafenib, and the subject formulation is used to treat arthrofibrosis.

In some embodiments, the subject formulation comprises ponatinib, and the subject formulation is used to treat arthrofibrosis.

In some embodiments, the subject formulation comprises pazopanib, and the subject formulation is used to treat arthrofibrosis.

In some embodiments, the subject formulation comprises axitinib, and the subject formulation is used to treat Peyronie's disease.

In some embodiments, the subject formulation comprises nintedanib, and the subject formulation is used to treat Peyronie's disease.

In some embodiments, the subject formulation comprises pirfenidone, and the subject formulation is used to treat Peyronie's disease.

In some embodiments, the subject formulation comprises riociguat, and the subject formulation is used to treat Peyronie's disease.

In some embodiments, the subject formulation comprises sorafenib, and the subject formulation is used to treat Peyronie's disease.

In some embodiments, the subject formulation comprises sunitinib, and the subject formulation is used to treat Peyronie's disease.

In some embodiments, the subject formulation comprises lenvatinib, and the subject formulation is used to treat Peyronie's disease.

In some embodiments, the subject formulation comprises regorafenib, and the subject formulation is used to treat Peyronie's disease.

In some embodiments, the subject formulation comprises ponatinib, and the subject formulation is used to treat Peyronie's disease.

In some embodiments, the subject formulation comprises pazopanib, and the subject formulation is used to treat Peyronie's disease.

In some embodiments, the subject formulation comprises axitinib, and the subject formulation is used to treat Dupuytren's contracture.

In some embodiments, the subject formulation comprises nintedanib, and the subject formulation is used to treat Dupuytren's contracture.

In some embodiments, the subject formulation comprises pirfenidone, and the subject formulation is used to treat Dupuytren's contracture.

In some embodiments, the subject formulation comprises riociguat, and the subject formulation is used to treat Dupuytren's contracture.

In some embodiments, the subject formulation comprises sorafenib, and the subject formulation is used to treat Dupuytren's contracture.

In some embodiments, the subject formulation comprises sunitinib, and the subject formulation is used to treat Dupuytren's contracture.

In some embodiments, the subject formulation comprises lenvatinib, and the subject formulation is used to treat Dupuytren's contracture.

In some embodiments, the subject formulation comprises regorafenib, and the subject formulation is used to treat Dupuytren's contracture.

In some embodiments, the subject formulation comprises ponatinib, and the subject formulation is used to treat Dupuytren's contracture.

In some embodiments, the subject formulation comprises pazopanib, and the subject formulation is used to treat Dupuytren's contracture.

In some embodiments, the subject formulation comprises axitinib, and the subject formulation is used to treat adhesive capsulitis.

In some embodiments, the subject formulation comprises nintedanib, and the subject formulation is used to treat adhesive capsulitis.

In some embodiments, the subject formulation comprises pirfenidone, and the subject formulation is used to treat adhesive capsulitis.

In some embodiments, the subject formulation comprises riociguat, and the subject formulation is used to treat adhesive capsulitis.

In some embodiments, the subject formulation comprises sorafenib, and the subject formulation is used to treat adhesive capsulitis.

In some embodiments, the subject formulation comprises sunitinib, and the subject formulation is used to treat adhesive capsulitis.

In some embodiments, the subject formulation comprises lenvatinib, and the subject formulation is used to treat adhesive capsulitis.

In some embodiments, the subject formulation comprises regorafenib, and the subject formulation is used to treat adhesive capsulitis.

In some embodiments, the subject formulation comprises ponatinib, and the subject formulation is used to treat adhesive capsulitis.

In some embodiments, the subject formulation comprises pazopanib, and the subject formulation is used to treat adhesive capsulitis.

In some embodiments, the subject formulation comprises axitinib, and the subject formulation is used to treat a choroidal neovascularization associated disease.

In some embodiments, the subject formulation comprises nintedanib, and the subject formulation is used to treat a choroidal neovascularization associated disease.

In some embodiments, the subject formulation comprises pirfenidone, and the subject formulation is used to treat a choroidal neovascularization associated disease.

In some embodiments, the subject formulation comprises riociguat, and the subject formulation is used to treat a choroidal neovascularization associated disease.

In some embodiments, the subject formulation comprises sorafenib, and the subject formulation is used to treat a choroidal neovascularization associated disease.

In some embodiments, the subject formulation comprises sunitinib, and the subject formulation is used to treat a choroidal neovascularization associated disease.

In some embodiments, the subject formulation comprises lenvatinib, and the subject formulation is used to treat a choroidal neovascularization associated disease.

In some embodiments, the subject formulation comprises regorafenib, and the subject formulation is used to treat a choroidal neovascularization associated disease.

In some embodiments, the subject formulation comprises ponatinib, and the subject formulation is used to treat a choroidal neovascularization associated disease.

In some embodiments, the subject formulation comprises pazopanib, and the subject formulation is used to treat a choroidal neovascularization associated disease.

In some embodiments, the subject formulation comprises axitinib, and the subject formulation is used to treat age-related macular degeneration.

In some embodiments, the subject formulation comprises nintedanib, and the subject formulation is used to treat age-related macular degeneration.

In some embodiments, the subject formulation comprises pirfenidone, and the subject formulation is used to treat age-related macular degeneration.

In some embodiments, the subject formulation comprises riociguat, and the subject formulation is used to treat age-related macular degeneration.

In some embodiments, the subject formulation comprises sorafenib, and the subject formulation is used to treat age-related macular degeneration.

In some embodiments, the subject formulation comprises sunitinib, and the subject formulation is used to treat age-related macular degeneration.

In some embodiments, the subject formulation comprises lenvatinib, and the subject formulation is used to treat age-related macular degeneration.

In some embodiments, the subject formulation comprises regorafenib, and the subject formulation is used to treat age-related macular degeneration.

In some embodiments, the subject formulation comprises ponatinib, and the subject formulation is used to treat age-related macular degeneration.

In some embodiments, the subject formulation comprises pazopanib, and the subject formulation is used to treat age-related macular degeneration.

In some embodiments, the subject formulation comprises axitinib, and the subject formulation is used to treat compromised corneal transparency.

In some embodiments, the subject formulation comprises nintedanib, and the subject formulation is used to treat compromised corneal transparency.

In some embodiments, the subject formulation comprises pirfenidone, and the subject formulation is used to treat compromised corneal transparency.

In some embodiments, the subject formulation comprises riociguat, and the subject formulation is used to treat compromised corneal transparency.

In some embodiments, the subject formulation comprises sorafenib, and the subject formulation is used to treat compromised corneal transparency.

In some embodiments, the subject formulation comprises sunitinib, and the subject formulation is used to treat compromised corneal transparency.

In some embodiments, the subject formulation comprises lenvatinib, and the subject formulation is used to treat compromised corneal transparency.

In some embodiments, the subject formulation comprises regorafenib, and the subject formulation is used to treat compromised corneal transparency.

In some embodiments, the subject formulation comprises ponatinib, and the subject formulation is used to treat compromised corneal transparency.

In some embodiments, the subject formulation comprises pazopanib, and the subject formulation is used to treat compromised corneal transparency.

In some embodiments, the subject formulation comprises axitinib, and the subject formulation is used to treat cornea scar formation.

In some embodiments, the subject formulation comprises nintedanib, and the subject formulation is used to treat cornea scar formation.

In some embodiments, the subject formulation comprises pirfenidone, and the subject formulation is used to treat cornea scar formation.

In some embodiments, the subject formulation comprises riociguat, and the subject formulation is used to treat cornea scar formation.

In some embodiments, the subject formulation comprises sorafenib, and the subject formulation is used to treat cornea scar formation.

In some embodiments, the subject formulation comprises sunitinib, and the subject formulation is used to treat cornea scar formation.

In some embodiments, the subject formulation comprises lenvatinib, and the subject formulation is used to treat cornea scar formation.

In some embodiments, the subject formulation comprises regorafenib, and the subject formulation is used to treat cornea scar formation.

In some embodiments, the subject formulation comprises ponatinib, and the subject formulation is used to treat cornea scar formation.

In some embodiments, the subject formulation comprises pazopanib, and the subject formulation is used to treat cornea scar formation.

In some embodiments, the subject formulation comprises axitinib, and the subject formulation is used to treat pterygia.

In some embodiments, the subject formulation comprises nintedanib, and the subject formulation is used to treat pterygia.

In some embodiments, the subject formulation comprises pirfenidone, and the subject formulation is used to treat pterygia.

In some embodiments, the subject formulation comprises riociguat, and the subject formulation is used to treat pterygia.

In some embodiments, the subject formulation comprises sorafenib, and the subject formulation is used to treat pterygia.

In some embodiments, the subject formulation comprises sunitinib, and the subject formulation is used to treat pterygia.

In some embodiments, the subject formulation comprises lenvatinib, and the subject formulation is used to treat pterygia.

In some embodiments, the subject formulation comprises regorafenib, and the subject formulation is used to treat pterygia.

In some embodiments, the subject formulation comprises ponatinib, and the subject formulation is used to treat pterygia.

In some embodiments, the subject formulation comprises pazopanib, and the subject formulation is used to treat pterygia.

In some embodiments, the subject formulation comprises axitinib, and the subject formulation is used to treat anterior cataract formation.

In some embodiments, the subject formulation comprises nintedanib, and the subject formulation is used to treat anterior cataract formation.

In some embodiments, the subject formulation comprises pirfenidone, and the subject formulation is used to treat anterior cataract formation.

In some embodiments, the subject formulation comprises riociguat, and the subject formulation is used to treat anterior cataract formation.

In some embodiments, the subject formulation comprises sorafenib, and the subject formulation is used to treat anterior cataract formation.

In some embodiments, the subject formulation comprises sunitinib, and the subject formulation is used to treat anterior cataract formation.

In some embodiments, the subject formulation comprises lenvatinib, and the subject formulation is used to treat anterior cataract formation.

In some embodiments, the subject formulation comprises regorafenib, and the subject formulation is used to treat anterior cataract formation.

In some embodiments, the subject formulation comprises ponatinib, and the subject formulation is used to treat anterior cataract formation.

In some embodiments, the subject formulation comprises pazopanib, and the subject formulation is used to treat anterior cataract formation.

In some embodiments, the subject formulation comprises axitinib, and the subject formulation is used to treat disorders related to glaucoma filtration surgery.

In some embodiments, the subject formulation comprises nintedanib, and the subject formulation is used to treat disorders related to glaucoma filtration surgery.

In some embodiments, the subject formulation comprises pirfenidone, and the subject formulation is used to treat disorders related to glaucoma filtration surgery.

In some embodiments, the subject formulation comprises riociguat, and the subject formulation is used to treat disorders related to glaucoma filtration surgery.

In some embodiments, the subject formulation comprises sorafenib, and the subject formulation is used to treat disorders related to glaucoma filtration surgery.

In some embodiments, the subject formulation comprises sunitinib, and the subject formulation is used to treat disorders related to glaucoma filtration surgery.

In some embodiments, the subject formulation comprises lenvatinib, and the subject formulation is used to treat disorders related to glaucoma filtration surgery.

In some embodiments, the subject formulation comprises regorafenib, and the subject formulation is used to treat disorders related to glaucoma filtration surgery.

In some embodiments, the subject formulation comprises ponatinib, and the subject formulation is used to treat disorders related to glaucoma filtration surgery.

In some embodiments, the subject formulation comprises pazopanib, and the subject formulation is used to treat disorders related to glaucoma filtration surgery.

In some embodiments, the subject formulation comprises axitinib, and the subject formulation is used to treat glaucoma.

In some embodiments, the subject formulation comprises nintedanib, and the subject formulation is used to treat glaucoma.

In some embodiments, the subject formulation comprises pirfenidone, and the subject formulation is used to treat glaucoma.

In some embodiments, the subject formulation comprises riociguat, and the subject formulation is used to treat glaucoma.

In some embodiments, the subject formulation comprises sorafenib, and the subject formulation is used to treat glaucoma.

In some embodiments, the subject formulation comprises sunitinib, and the subject formulation is used to treat glaucoma.

In some embodiments, the subject formulation comprises lenvatinib, and the subject formulation is used to treat glaucoma.

In some embodiments, the subject formulation comprises regorafenib, and the subject formulation is used to treat glaucoma.

In some embodiments, the subject formulation comprises ponatinib, and the subject formulation is used to treat glaucoma.

In some embodiments, the subject formulation comprises pazopanib, and the subject formulation is used to treat glaucoma.

In some embodiments, the subject formulation comprises axitinib, and the subject formulation is used to treat a condition related to photorefractive keratectomy.

In some embodiments, the subject formulation comprises nintedanib, and the subject formulation is used to treat a condition related to photorefractive keratectomy.

In some embodiments, the subject formulation comprises pirfenidone, and the subject formulation is used to treat a condition related to photorefractive keratectomy.

In some embodiments, the subject formulation comprises riociguat, and the subject formulation is used to treat a condition related to photorefractive keratectomy.

In some embodiments, the subject formulation comprises sorafenib, and the subject formulation is used to treat a condition related to photorefractive keratectomy.

In some embodiments, the subject formulation comprises sunitinib, and the subject formulation is used to treat a condition related to photorefractive keratectomy.

In some embodiments, the subject formulation comprises lenvatinib, and the subject formulation is used to treat a condition related to photorefractive keratectomy.

In some embodiments, the subject formulation comprises regorafenib, and the subject formulation is used to treat a condition related to photorefractive keratectomy.

In some embodiments, the subject formulation comprises ponatinib, and the subject formulation is used to treat a condition related to photorefractive keratectomy.

In some embodiments, the subject formulation comprises pazopanib, and the subject formulation is used to treat a condition related to photorefractive keratectomy.

In some embodiments, the subject formulation comprises axitinib, and the subject formulation is used to treat a disorder related to laser in situ keratomileusis.

In some embodiments, the subject formulation comprises nintedanib, and the subject formulation is used to treat a disorder related to laser in situ keratomileusis.

In some embodiments, the subject formulation comprises pirfenidone, and the subject formulation is used to treat a disorder related to laser in situ keratomileusis.

In some embodiments, the subject formulation comprises riociguat, and the subject formulation is used to treat a disorder related to laser in situ keratomileusis.

In some embodiments, the subject formulation comprises sorafenib, and the subject formulation is used to treat a disorder related to laser in situ keratomileusis.

In some embodiments, the subject formulation comprises sunitinib, and the subject formulation is used to treat a disorder related to laser in situ keratomileusis.

In some embodiments, the subject formulation comprises lenvatinib, and the subject formulation is used to treat a disorder related to laser in situ keratomileusis.

In some embodiments, the subject formulation comprises regorafenib, and the subject formulation is used to treat a disorder related to laser in situ keratomileusis.

In some embodiments, the subject formulation comprises ponatinib, and the subject formulation is used to treat a disorder related to laser in situ keratomileusis.

In some embodiments, the subject formulation comprises pazopanib, and the subject formulation is used to treat a disorder related to laser in situ keratomileusis.

In some embodiments, the subject formulation comprises axitinib, and the subject formulation is used to treat a disorder related to contraction of the pre- and/or epiretinal membranes.

In some embodiments, the subject formulation comprises nintedanib, and the subject formulation is used to treat a disorder related to contraction of the pre- and/or epiretinal membranes.

In some embodiments, the subject formulation comprises pirfenidone, and the subject formulation is used to treat a disorder related to contraction of the pre- and/or epiretinal membranes.

In some embodiments, the subject formulation comprises riociguat, and the subject formulation is used to treat a disorder related to contraction of the pre- and/or epiretinal membranes.

In some embodiments, the subject formulation comprises sorafenib, and the subject formulation is used to treat a disorder related to contraction of the pre- and/or epiretinal membranes.

In some embodiments, the subject formulation comprises sunitinib, and the subject formulation is used to treat a disorder related to contraction of the pre- and/or epiretinal membranes.

In some embodiments, the subject formulation comprises lenvatinib, and the subject formulation is used to treat a disorder related to contraction of the pre- and/or epiretinal membranes.

In some embodiments, the subject formulation comprises regorafenib, and the subject formulation is used to treat a disorder related to contraction of the pre- and/or epiretinal membranes.

In some embodiments, the subject formulation comprises ponatinib, and the subject formulation is used to treat a disorder related to contraction of the pre- and/or epiretinal membranes.

In some embodiments, the subject formulation comprises pazopanib, and the subject formulation is used to treat a disorder related to contraction of the pre- and/or epiretinal membranes.

In some embodiments, the subject formulation comprises axitinib, and the subject formulation is used to treat proliferative vitreoretinopathy.

In some embodiments, the subject formulation comprises nintedanib, and the subject formulation is used to treat proliferative vitreoretinopathy.

In some embodiments, the subject formulation comprises pirfenidone, and the subject formulation is used to treat proliferative vitreoretinopathy.

In some embodiments, the subject formulation comprises riociguat, and the subject formulation is used to treat proliferative vitreoretinopathy.

In some embodiments, the subject formulation comprises sorafenib, and the subject formulation is used to treat proliferative vitreoretinopathy.

In some embodiments, the subject formulation comprises sunitinib, and the subject formulation is used to treat proliferative vitreoretinopathy.

In some embodiments, the subject formulation comprises lenvatinib, and the subject formulation is used to treat proliferative vitreoretinopathy.

In some embodiments, the subject formulation comprises regorafenib, and the subject formulation is used to treat proliferative vitreoretinopathy.

In some embodiments, the subject formulation comprises ponatinib, and the subject formulation is used to treat proliferative vitreoretinopathy.

In some embodiments, the subject formulation comprises pazopanib, and the subject formulation is used to treat proliferative vitreoretinopathy.

In some embodiments, the subject formulation comprises axitinib, and the subject formulation is used to treat proliferative diabetic retinopathy.

In some embodiments, the subject formulation comprises nintedanib, and the subject formulation is used to treat proliferative diabetic retinopathy.

In some embodiments, the subject formulation comprises pirfenidone, and the subject formulation is used to treat proliferative diabetic retinopathy.

In some embodiments, the subject formulation comprises riociguat, and the subject formulation is used to treat proliferative diabetic retinopathy.

In some embodiments, the subject formulation comprises sorafenib, and the subject formulation is used to treat proliferative diabetic retinopathy.

In some embodiments, the subject formulation comprises sunitinib, and the subject formulation is used to treat proliferative diabetic retinopathy.

In some embodiments, the subject formulation comprises lenvatinib, and the subject formulation is used to treat proliferative diabetic retinopathy.

In some embodiments, the subject formulation comprises regorafenib, and the subject formulation is used to treat proliferative diabetic retinopathy.

In some embodiments, the subject formulation comprises ponatinib, and the subject formulation is used to treat proliferative diabetic retinopathy.

In some embodiments, the subject formulation comprises pazopanib, and the subject formulation is used to treat proliferative diabetic retinopathy.

In some embodiments, the subject formulation comprises axitinib, and the subject formulation is used to treat diabetic macular edema.

In some embodiments, the subject formulation comprises nintedanib, and the subject formulation is used to treat diabetic macular edema.

In some embodiments, the subject formulation comprises pirfenidone, and the subject formulation is used to treat diabetic macular edema.

In some embodiments, the subject formulation comprises riociguat, and the subject formulation is used to treat diabetic macular edema.

In some embodiments, the subject formulation comprises sorafenib, and the subject formulation is used to treat diabetic macular edema.

In some embodiments, the subject formulation comprises sunitinib, and the subject formulation is used to treat diabetic macular edema.

In some embodiments, the subject formulation comprises lenvatinib, and the subject formulation is used to treat diabetic macular edema.

In some embodiments, the subject formulation comprises regorafenib, and the subject formulation is used to treat diabetic macular edema.

In some embodiments, the subject formulation comprises ponatinib, and the subject formulation is used to treat diabetic macular edema.

In some embodiments, the subject formulation comprises pazopanib, and the subject formulation is used to treat diabetic macular edema.

In some embodiments, the subject formulation comprises axitinib, and the subject formulation is used to treat disorders related to myopic choroidal neovascularization.

In some embodiments, the subject formulation comprises nintedanib, and the subject formulation is used to treat disorders related to myopic choroidal neovascularization.

In some embodiments, the subject formulation comprises pirfenidone, and the subject formulation is used to treat disorders related to myopic choroidal neovascularization.

In some embodiments, the subject formulation comprises riociguat, and the subject formulation is used to treat disorders related to myopic choroidal neovascularization.

In some embodiments, the subject formulation comprises sorafenib, and the subject formulation is used to treat disorders related to myopic choroidal neovascularization.

In some embodiments, the subject formulation comprises sunitinib, and the subject formulation is used to treat disorders related to myopic choroidal neovascularization.

In some embodiments, the subject formulation comprises lenvatinib, and the subject formulation is used to treat disorders related to myopic choroidal neovascularization.

In some embodiments, the subject formulation comprises regorafenib, and the subject formulation is used to treat disorders related to myopic choroidal neovascularization.

In some embodiments, the subject formulation comprises ponatinib, and the subject formulation is used to treat disorders related to myopic choroidal neovascularization.

In some embodiments, the subject formulation comprises pazopanib, and the subject formulation is used to treat disorders related to myopic choroidal neovascularization.

In some embodiments, the subject formulation comprises axitinib, and the subject formulation is used to treat retinal vein occlusion.

In some embodiments, the subject formulation comprises nintedanib, and the subject formulation is used to treat retinal vein occlusion.

In some embodiments, the subject formulation comprises pirfenidone, and the subject formulation is used to treat retinal vein occlusion.

In some embodiments, the subject formulation comprises riociguat, and the subject formulation is used to treat retinal vein occlusion.

In some embodiments, the subject formulation comprises sorafenib, and the subject formulation is used to treat retinal vein occlusion.

In some embodiments, the subject formulation comprises sunitinib, and the subject formulation is used to treat retinal vein occlusion.

In some embodiments, the subject formulation comprises lenvatinib, and the subject formulation is used to treat retinal vein occlusion.

In some embodiments, the subject formulation comprises regorafenib, and the subject formulation is used to treat retinal vein occlusion.

In some embodiments, the subject formulation comprises ponatinib, and the subject formulation is used to treat retinal vein occlusion.

In some embodiments, the subject formulation comprises pazopanib, and the subject formulation is used to treat retinal vein occlusion.

In some embodiments, the subject formulation comprises axitinib, and the subject formulation is used to treat sub-retinal fibrosis.

In some embodiments, the subject formulation comprises nintedanib, and the subject formulation is used to treat sub-retinal fibrosis.

In some embodiments, the subject formulation comprises pirfenidone, and the subject formulation is used to treat sub-retinal fibrosis.

In some embodiments, the subject formulation comprises riociguat, and the subject formulation is used to treat sub-retinal fibrosis.

In some embodiments, the subject formulation comprises sorafenib, and the subject formulation is used to treat sub-retinal fibrosis.

In some embodiments, the subject formulation comprises sunitinib, and the subject formulation is used to treat sub-retinal fibrosis.

In some embodiments, the subject formulation comprises lenvatinib, and the subject formulation is used to treat sub-retinal fibrosis.

In some embodiments, the subject formulation comprises regorafenib, and the subject formulation is used to treat sub-retinal fibrosis.

In some embodiments, the subject formulation comprises ponatinib, and the subject formulation is used to treat sub-retinal fibrosis.

In some embodiments, the subject formulation comprises pazopanib, and the subject formulation is used to treat sub-retinal fibrosis.

In some embodiments, the subject formulation comprises axitinib, and the subject formulation is used to treat sub-retinal scarring.

In some embodiments, the subject formulation comprises nintedanib, and the subject formulation is used to treat sub-retinal scarring.

In some embodiments, the subject formulation comprises pirfenidone, and the subject formulation is used to treat sub-retinal scarring.

In some embodiments, the subject formulation comprises riociguat, and the subject formulation is used to treat sub-retinal scarring.

In some embodiments, the subject formulation comprises sorafenib, and the subject formulation is used to treat sub-retinal scarring.

In some embodiments, the subject formulation comprises sunitinib, and the subject formulation is used to treat sub-retinal scarring.

In some embodiments, the subject formulation comprises lenvatinib, and the subject formulation is used to treat sub-retinal scarring.

In some embodiments, the subject formulation comprises regorafenib, and the subject formulation is used to treat sub-retinal scarring.

In some embodiments, the subject formulation comprises ponatinib, and the subject formulation is used to treat sub-retinal scarring.

In some embodiments, the subject formulation comprises pazopanib, and the subject formulation is used to treat sub-retinal scarring.

In some embodiments, the subject formulation comprises axitinib, and the subject formulation is used to treat choroidal membrane related disorders.

In some embodiments, the subject formulation comprises nintedanib, and the subject formulation is used to treat choroidal membrane related disorders.

In some embodiments, the subject formulation comprises pirfenidone, and the subject formulation is used to treat choroidal membrane related disorders.

In some embodiments, the subject formulation comprises riociguat, and the subject formulation is used to treat choroidal membrane related disorders.

In some embodiments, the subject formulation comprises sorafenib, and the subject formulation is used to treat choroidal membrane related disorders.

In some embodiments, the subject formulation comprises sunitinib, and the subject formulation is used to treat choroidal membrane related disorders.

In some embodiments, the subject formulation comprises lenvatinib, and the subject formulation is used to treat choroidal membrane related disorders.

In some embodiments, the subject formulation comprises regorafenib, and the subject formulation is used to treat choroidal membrane related disorders.

In some embodiments, the subject formulation comprises ponatinib, and the subject formulation is used to treat choroidal membrane related disorders.

In some embodiments, the subject formulation comprises pazopanib, and the subject formulation is used to treat choroidal membrane related disorders.

In some embodiments, the subject formulation comprises axitinib, and the subject formulation is used to treat retinal gliosis.

In some embodiments, the subject formulation comprises nintedanib, and the subject formulation is used to treat retinal gliosis.

In some embodiments, the subject formulation comprises pirfenidone, and the subject formulation is used to treat retinal gliosis.

In some embodiments, the subject formulation comprises riociguat, and the subject formulation is used to treat retinal gliosis.

In some embodiments, the subject formulation comprises sorafenib, and the subject formulation is used to treat retinal gliosis.

In some embodiments, the subject formulation comprises sunitinib, and the subject formulation is used to treat retinal gliosis.

In some embodiments, the subject formulation comprises lenvatinib, and the subject formulation is used to treat retinal gliosis.

In some embodiments, the subject formulation comprises regorafenib, and the subject formulation is used to treat retinal gliosis.

In some embodiments, the subject formulation comprises ponatinib, and the subject formulation is used to treat retinal gliosis.

In some embodiments, the subject formulation comprises pazopanib, and the subject formulation is used to treat retinal gliosis.

In some embodiments, the subject formulation comprises axitinib, and the subject formulation is used to treat an epiretinal membrane related disorder.

In some embodiments, the subject formulation comprises nintedanib, and the subject formulation is used to treat an epiretinal membrane related disorder.

In some embodiments, the subject formulation comprises pirfenidone, and the subject formulation is used to treat an epiretinal membrane related disorder.

In some embodiments, the subject formulation comprises riociguat, and the subject formulation is used to treat an epiretinal membrane related disorder.

In some embodiments, the subject formulation comprises sorafenib, and the subject formulation is used to treat an epiretinal membrane related disorder.

In some embodiments, the subject formulation comprises sunitinib, and the subject formulation is used to treat an epiretinal membrane related disorder.

In some embodiments, the subject formulation comprises lenvatinib, and the subject formulation is used to treat an epiretinal membrane related disorder.

In some embodiments, the subject formulation comprises regorafenib, and the subject formulation is used to treat an epiretinal membrane related disorder.

In some embodiments, the subject formulation comprises ponatinib, and the subject formulation is used to treat an epiretinal membrane related disorder.

In some embodiments, the subject formulation comprises pazopanib, and the subject formulation is used to treat an epiretinal membrane related disorder.

In some embodiments, the subject formulation comprises axitinib, and the subject formulation is used to treat glial scar formation.

In some embodiments, the subject formulation comprises nintedanib, and the subject formulation is used to treat glial scar formation.

In some embodiments, the subject formulation comprises pirfenidone, and the subject formulation is used to treat glial scar formation.

In some embodiments, the subject formulation comprises riociguat, and the subject formulation is used to treat glial scar formation.

In some embodiments, the subject formulation comprises sorafenib, and the subject formulation is used to treat glial scar formation.

In some embodiments, the subject formulation comprises sunitinib, and the subject formulation is used to treat glial scar formation.

In some embodiments, the subject formulation comprises lenvatinib, and the subject formulation is used to treat glial scar formation.

In some embodiments, the subject formulation comprises regorafenib, and the subject formulation is used to treat glial scar formation.

In some embodiments, the subject formulation comprises ponatinib, and the subject formulation is used to treat glial scar formation.

In some embodiments, the subject formulation comprises pazopanib, and the subject formulation is used to treat glial scar formation.

In some embodiments, the subject formulation comprises axitinib, and the subject formulation is used to treat benign prostate hyperplasia.

In some embodiments, the subject formulation comprises nintedanib, and the subject formulation is used to treat benign prostate hyperplasia.

In some embodiments, the subject formulation comprises pirfenidone, and the subject formulation is used to treat benign prostate hyperplasia.

In some embodiments, the subject formulation comprises riociguat, and the subject formulation is used to treat benign prostate hyperplasia.

In some embodiments, the subject formulation comprises sorafenib, and the subject formulation is used to treat benign prostate hyperplasia.

In some embodiments, the subject formulation comprises sunitinib, and the subject formulation is used to treat benign prostate hyperplasia.

In some embodiments, the subject formulation comprises lenvatinib, and the subject formulation is used to treat benign prostate hyperplasia.

In some embodiments, the subject formulation comprises regorafenib, and the subject formulation is used to treat benign prostate hyperplasia.

In some embodiments, the subject formulation comprises ponatinib, and the subject formulation is used to treat benign prostate hyperplasia.

In some embodiments, the subject formulation comprises pazopanib, and the subject formulation is used to treat benign prostate hyperplasia.

In some embodiments, the subject formulation comprises axitinib, and the subject formulation is used to treat a lower urinary tract symptom.

In some embodiments, the subject formulation comprises nintedanib, and the subject formulation is used to treat a lower urinary tract symptom.

In some embodiments, the subject formulation comprises pirfenidone, and the subject formulation is used to treat a lower urinary tract symptom.

In some embodiments, the subject formulation comprises riociguat, and the subject formulation is used to treat a lower urinary tract symptom.

In some embodiments, the subject formulation comprises sorafenib, and the subject formulation is used to treat a lower urinary tract symptom.

In some embodiments, the subject formulation comprises sunitinib, and the subject formulation is used to treat a lower urinary tract symptom.

In some embodiments, the subject formulation comprises lenvatinib, and the subject formulation is used to treat a lower urinary tract symptom.

In some embodiments, the subject formulation comprises regorafenib, and the subject formulation is used to treat a lower urinary tract symptom.

In some embodiments, the subject formulation comprises ponatinib, and the subject formulation is used to treat a lower urinary tract symptom.

In some embodiments, the subject formulation comprises pazopanib, and the subject formulation is used to treat a lower urinary tract symptom.

In some embodiments, the subject formulation comprises axitinib, and the subject formulation is used to treat benign prostatic enlargement.

In some embodiments, the subject formulation comprises nintedanib, and the subject formulation is used to treat benign prostatic enlargement.

In some embodiments, the subject formulation comprises pirfenidone, and the subject formulation is used to treat benign prostatic enlargement.

In some embodiments, the subject formulation comprises riociguat, and the subject formulation is used to treat benign prostatic enlargement.

In some embodiments, the subject formulation comprises sorafenib, and the subject formulation is used to treat benign prostatic enlargement.

In some embodiments, the subject formulation comprises sunitinib, and the subject formulation is used to treat benign prostatic enlargement.

In some embodiments, the subject formulation comprises lenvatinib, and the subject formulation is used to treat benign prostatic enlargement.

In some embodiments, the subject formulation comprises regorafenib, and the subject formulation is used to treat benign prostatic enlargement.

In some embodiments, the subject formulation comprises ponatinib, and the subject formulation is used to treat benign prostatic enlargement.

In some embodiments, the subject formulation comprises pazopanib, and the subject formulation is used to treat benign prostatic enlargement.

In some embodiments, the subject formulation comprises axitinib, and the subject formulation is used to treat bladder outlet obstruction.

In some embodiments, the subject formulation comprises nintedanib, and the subject formulation is used to treat bladder outlet obstruction.

In some embodiments, the subject formulation comprises pirfenidone, and the subject formulation is used to treat bladder outlet obstruction.

In some embodiments, the subject formulation comprises riociguat, and the subject formulation is used to treat bladder outlet obstruction.

In some embodiments, the subject formulation comprises sorafenib, and the subject formulation is used to treat bladder outlet obstruction.

In some embodiments, the subject formulation comprises sunitinib, and the subject formulation is used to treat bladder outlet obstruction.

In some embodiments, the subject formulation comprises lenvatinib, and the subject formulation is used to treat bladder outlet obstruction.

In some embodiments, the subject formulation comprises regorafenib, and the subject formulation is used to treat bladder outlet obstruction.

In some embodiments, the subject formulation comprises ponatinib, and the subject formulation is used to treat bladder outlet obstruction.

In some embodiments, the subject formulation comprises pazopanib, and the subject formulation is used to treat bladder outlet obstruction.

In some embodiments, the subject formulation comprises axitinib, and the subject formulation is used to treat an overactive bladder-related disorder.

In some embodiments, the subject formulation comprises nintedanib, and the subject formulation is used to treat an overactive bladder-related disorder.

In some embodiments, the subject formulation comprises pirfenidone, and the subject formulation is used to treat an overactive bladder-related disorder.

In some embodiments, the subject formulation comprises riociguat, and the subject formulation is used to treat an overactive bladder-related disorder.

In some embodiments, the subject formulation comprises sorafenib, and the subject formulation is used to treat an overactive bladder-related disorder.

In some embodiments, the subject formulation comprises sunitinib, and the subject formulation is used to treat an overactive bladder-related disorder.

In some embodiments, the subject formulation comprises lenvatinib, and the subject formulation is used to treat an overactive bladder-related disorder.

In some embodiments, the subject formulation comprises regorafenib, and the subject formulation is used to treat an overactive bladder-related disorder.

In some embodiments, the subject formulation comprises ponatinib, and the subject formulation is used to treat an overactive bladder-related disorder.

In some embodiments, the subject formulation comprises pazopanib, and the subject formulation is used to treat an overactive bladder-related disorder.

In some embodiments, the subject formulation comprises axitinib, and the subject formulation is used to treat prostatitis.

In some embodiments, the subject formulation comprises nintedanib, and the subject formulation is used to treat prostatitis.

In some embodiments, the subject formulation comprises pirfenidone, and the subject formulation is used to treat prostatitis.

In some embodiments, the subject formulation comprises riociguat, and the subject formulation is used to treat prostatitis.

In some embodiments, the subject formulation comprises sorafenib, and the subject formulation is used to treat prostatitis.

In some embodiments, the subject formulation comprises sunitinib, and the subject formulation is used to treat prostatitis.

In some embodiments, the subject formulation comprises lenvatinib, and the subject formulation is used to treat prostatitis.

In some embodiments, the subject formulation comprises regorafenib, and the subject formulation is used to treat prostatitis.

In some embodiments, the subject formulation comprises ponatinib, and the subject formulation is used to treat prostatitis.

In some embodiments, the subject formulation comprises pazopanib, and the subject formulation is used to treat prostatitis.

In some embodiments, the subject formulation comprises axitinib, and the subject formulation is used to treat prostatic intraepithelial neoplasia.

In some embodiments, the subject formulation comprises nintedanib, and the subject formulation is used to treat prostatic intraepithelial neoplasia.

In some embodiments, the subject formulation comprises pirfenidone, and the subject formulation is used to treat prostatic intraepithelial neoplasia.

In some embodiments, the subject formulation comprises riociguat, and the subject formulation is used to treat prostatic intraepithelial neoplasia.

In some embodiments, the subject formulation comprises sorafenib, and the subject formulation is used to treat prostatic intraepithelial neoplasia.

In some embodiments, the subject formulation comprises sunitinib, and the subject formulation is used to treat prostatic intraepithelial neoplasia.

In some embodiments, the subject formulation comprises lenvatinib, and the subject formulation is used to treat prostatic intraepithelial neoplasia.

In some embodiments, the subject formulation comprises regorafenib, and the subject formulation is used to treat prostatic intraepithelial neoplasia.

In some embodiments, the subject formulation comprises ponatinib, and the subject formulation is used to treat prostatic intraepithelial neoplasia.

In some embodiments, the subject formulation comprises pazopanib, and the subject formulation is used to treat prostatic intraepithelial neoplasia.

In some embodiments, the subject formulation comprises axitinib, and the subject formulation is used to treat neurogenic bladder syndrome.

In some embodiments, the subject formulation comprises nintedanib, and the subject formulation is used to treat neurogenic bladder syndrome.

In some embodiments, the subject formulation comprises pirfenidone, and the subject formulation is used to treat neurogenic bladder syndrome.

In some embodiments, the subject formulation comprises riociguat, and the subject formulation is used to treat neurogenic bladder syndrome.

In some embodiments, the subject formulation comprises sorafenib, and the subject formulation is used to treat neurogenic bladder syndrome.

In some embodiments, the subject formulation comprises sunitinib, and the subject formulation is used to treat neurogenic bladder syndrome.

In some embodiments, the subject formulation comprises lenvatinib, and the subject formulation is used to treat neurogenic bladder syndrome.

In some embodiments, the subject formulation comprises regorafenib, and the subject formulation is used to treat neurogenic bladder syndrome.

In some embodiments, the subject formulation comprises ponatinib, and the subject formulation is used to treat neurogenic bladder syndrome.

In some embodiments, the subject formulation comprises pazopanib, and the subject formulation is used to treat neurogenic bladder syndrome.

In some embodiments, the subject formulation comprises axitinib, and the subject formulation is used to treat prostate cancer.

In some embodiments, the subject formulation comprises nintedanib, and the subject formulation is used to treat prostate cancer.

In some embodiments, the subject formulation comprises pirfenidone, and the subject formulation is used to treat prostate cancer.

In some embodiments, the subject formulation comprises riociguat, and the subject formulation is used to treat prostate cancer.

In some embodiments, the subject formulation comprises sorafenib, and the subject formulation is used to treat prostate cancer.

In some embodiments, the subject formulation comprises sunitinib, and the subject formulation is used to treat prostate cancer.

In some embodiments, the subject formulation comprises lenvatinib, and the subject formulation is used to treat prostate cancer.

In some embodiments, the subject formulation comprises regorafenib, and the subject formulation is used to treat prostate cancer.

In some embodiments, the subject formulation comprises ponatinib, and the subject formulation is used to treat prostate cancer.

In some embodiments, the subject formulation comprises pazopanib, and the subject formulation is used to treat prostate cancer.

In some embodiments, the subject formulation comprises axitinib, and the subject formulation is used to treat urinary incontinence.

In some embodiments, the subject formulation comprises nintedanib, and the subject formulation is used to treat urinary incontinence.

In some embodiments, the subject formulation comprises pirfenidone, and the subject formulation is used to treat urinary incontinence.

In some embodiments, the subject formulation comprises riociguat, and the subject formulation is used to treat urinary incontinence.

In some embodiments, the subject formulation comprises sorafenib, and the subject formulation is used to treat urinary incontinence.

In some embodiments, the subject formulation comprises sunitinib, and the subject formulation is used to treat urinary incontinence.

In some embodiments, the subject formulation comprises lenvatinib, and the subject formulation is used to treat urinary incontinence.

In some embodiments, the subject formulation comprises regorafenib, and the subject formulation is used to treat urinary incontinence.

In some embodiments, the subject formulation comprises ponatinib, and the subject formulation is used to treat urinary incontinence.

In some embodiments, the subject formulation comprises pazopanib, and the subject formulation is used to treat urinary incontinence.

In some embodiments, the subject formulation comprises axitinib, and the subject formulation is used to treat pelvic pain.

In some embodiments, the subject formulation comprises nintedanib, and the subject formulation is used to treat pelvic pain.

In some embodiments, the subject formulation comprises pirfenidone, and the subject formulation is used to treat pelvic pain.

In some embodiments, the subject formulation comprises riociguat, and the subject formulation is used to treat pelvic pain.

In some embodiments, the subject formulation comprises sorafenib, and the subject formulation is used to treat pelvic pain.

In some embodiments, the subject formulation comprises sunitinib, and the subject formulation is used to treat pelvic pain.

In some embodiments, the subject formulation comprises lenvatinib, and the subject formulation is used to treat pelvic pain.

In some embodiments, the subject formulation comprises regorafenib, and the subject formulation is used to treat pelvic pain.

In some embodiments, the subject formulation comprises ponatinib, and the subject formulation is used to treat pelvic pain.

In some embodiments, the subject formulation comprises pazopanib, and the subject formulation is used to treat pelvic pain.

Tables 2-4 are non-limiting examples of some subject formulations:

TABLE 2
Suspension Composition for Multi-target Inhibitors

| Ingredients | Grade | Conc. (w/w) |
|---|---|---|
| Multi-target inhibitor | n/a | 1-8% or 0.01-3% |
| Suspending agent, e.g. | USP | 0.1-10% or 0.2-3%, |
| carboxymethylcellulose, | | (e.g., 0.5%) |
| cellulose, or a mixture of | | |
| cellulose | | |
| Wetting agent, e.g. a | NF | 0.02-5%, |
| surfactant | | (e.g., 0.05%) |
| Buffer system | USP | 0.2-0.4% |
| Sodium chloride | USP | 0.4-0.6% |
| 1N sodium hydroxide | NF | adj. pH to 7.4 |
| Water for injection | USP | qs |

TABLE 3
Suspension Composition for Multi-target Inhibitors

| Ingredients | Grade | Conc. (w/w) |
|---|---|---|
| Multi-target inhibitor | n/a | 0.01-3% |
| Sodium | USP | 0.1-10% or 0.2-3%, |
| carboxymethylcellulose, | | (e.g., 0.5%) |
| low viscosity | | |
| Polysorbate 80 | NF | 0.02-5%, |
| | | (e.g., 0.05%) |
| Sodium phosphate dibasic | USP | 0.27% |
| heptahydrate | | |
| Sodium phosphate | USP | 0.03% |
| monobasic monohydrate | | |
| Sodium chloride | USP | 0.5% |
| 1N sodium hydroxide | NF | adj. pH to 7.4 |
| Water for injection | USP | qs |

TABLE 4
Suspension Composition for Multi-target Inhibitors

| Ingredients | Grade | Conc. (w/w) |
|---|---|---|
| Multi-target inhibitor | n/a | 0.01-3% |
| Sodium | USP | 1% |
| carboxymethylcellulose, low | | |
| viscosity | | |
| Polysorbate 80 | NF | 0.05% |
| Sodium phosphate dibasic | USP | 0.27% |
| heptahydrate | | |
| Sodium phosphate monobasic | USP | 0.03% |
| monohydrate | | |
| Sodium chloride | USP | 0.75% |
| 1N sodium hydroxide | NF | adj. pH to 7.4 |
| Water for injection | USP | qs |

TABLE 5
Suspension Composition for Multi-target Inhibitors

| Ingredients | Grade | Conc. (w/w) |
|---|---|---|
| Multi-target inhibitor | n/a | 0.01-10%, |
| | | preferably 0.01-3% |
| Sodium | USP | 1% |
| carboxymethylcellulose, | | |
| low viscosity | | |
| Polysorbate 80 | NF | 0.05% |
| Sodium phosphate dibasic | USP | 0.27% |
| heptahydrate | | |
| Sodium phosphate | USP | 0.03% |
| monobasic monohydrate | | |
| Sodium chloride | USP | 0.75% |
| Benzyl alcohol | NF | 0.5-2% (e.g., 0.9%) |
| 1N sodium hydroxide | NF | adj. pH to 7.4 |
| Water for injection | USP | qs |

Examples 1-3 depict the results of physical testing of some subject formulations.

### Example 1

Multi-Target Inhibitor Suspensions:
Particle Size, pH, Osmolality, and Syringeability

| Multi- | API Raw | Formulated Suspension | | | |
|---|---|---|---|---|---|
| Target | Material | Particle size | | | |
| Inhibitors | PSD* | distribution | pH | Osmolality | Syringeability |
| Axitinib | D90: 9.9 | D90: 15.6 | 7.6 | 206 | Pass 25G |
| | D50: 5.5 | D50: 5.8 | | | |
| Nintedanib | D90: 105 | D90: 35 | 7.6 | 207 | Pass 23G |
| | D50: 68 | D50: 22 | | | |
| Pirfenidone | D90: 74 | D90: 72 | 7.6 | 312 | Pass 23G |
| | D50: 57 | D50: 44 | | | |
| Riociguat | D90: 29 | D90: 17 | 7.6 | 201 | Pass 27G |
| | D50: 16 | D50: 9 | | | |
| Sorafenib | D90: 39 | D90: 20 | 7.3 | 216 | Pass 27G |
| Hemi | D50: 18 | D50: 11 | | | |
| Tosylate | | | | | |
| Sunitinib | D90: 52 | D90: 26 | 7.6 | 201 | Pass 25G |
| | D50: 25 | D50: 12 | | | |
| Lenvatinib | D90: 346 | D90: 25 | 7.6 | 201 | Pass 27G |
| | D50: 164 | D50: 7 | | | |

*PSD: particle size distribution; D90 is the diameter of the particle that 90% of a sample's mass is smaller than; D50 is the diameter of the particle that 50% of a sample's mass is smaller than.

### Example 2

Multi-Target Inhibitor Suspensions:
Terminal Sterilization Methods and Stability

| | | Assay (% of labeled claim) | | |
|---|---|---|---|---|
| | | of Formulated Suspension | | |
| Multi- | Terminal | | 6 weeks | 4 months |
| Target | Sterilization | | (40° C./ | (40° C./ |
| Inhibitors | Method | Initial | 75% RH) | 75% RH) |
| Axitinib | Gamma | 102.0 | 97.8 | 95.2 |
| | irradiation | 100.7 | 98.5 | 100.9 |
| | Autoclave | 98.7 | Not tested | 101.2 |
| | | (top) | | (top)^{#} |
| | | 100.1 | | 103.2 |
| | | (bottom) | | (bottom)^{#} |
| Nintedanib | Gamma | 101.8 | 98.1 | 90.0 |
| | irradiation | 103.0 | 99.7 | 97.9 |
| | Autoclave | 96.0 | Not tested | Not tested |
| Pirfenidone | Gamma | 90.9 | 89.9 | 91.5 |
| | irradiation | 97.2 | 95.7 | 94.9 |
| | Autoclave | Not tested | Not tested | Not tested |
| Riociguat | Gamma | 101.9 | 98.7 | 99.9 |
| | irradiation | 100.6 | 98.0 | 99.7 |
| | Autoclave | Not tested | Not tested | Not tested |
| Sorafenib | Gamma | 98.9 | 100.7 | 99.9 |
| Hemi | irradiation | 101.6 | 100.4 | 99.6 |
| Tosylate | Autoclave | Not tested | Not tested | Not tested |
| Sunitinib | Gamma | 96.7 | 98.9 | 100.0 |
| | irradiation | 95.1 | 96.5 | 95.6 |
| | Autoclave | Not tested | Not tested | Not tested |
| Lenvatinib | Gamma | 101.4 | 99.2 | 99.8 |
| | irradiation | 102.4 | 99.8 | 100.2 |
| | Autoclave | 99.0 | Not tested | Not tested |

^{#}tested at 3 months instead of 4 months.

Some pharmaceutical compositions described herein been shown to deliver therapeutically effective concentrations in local tissues, which are maintained over prolonged periods of time. These prolonged tissue exposures would not be possible based on the systemic half-lives and clearance rates of these active ingredients. For example, the terminal plasma half-life in man was 2-5 hours for axitinib (Rugo et al, 2005), 9.5 hours for nintedanib (US label), 30 hours for lenvatinib (Yamada 2011), and 28 hours for regorafenib (US label).

### Example 3

The tolerability, efficacy, and ocular distribution were evaluated in a laser-induced choroid neovascularization model in rats. Good efficacy and high tissue concentrations in were seen in the posterior segment of the rat eye twenty-two days after one intravitreal injection or three times daily eyedrop instillation in the rat eyes.

In this 22-day study, effects of local administration of the multi-target (i.e., multi-target kinase) inhibitors nintedanib and lenvatinib were examined on the development of new vessels. Vehicle and positive control groups were included in the study. Female Brown Norway rats were divided into 7 separate treatment groups. On Day 1, laser treatments were performed on all animals using a 520 nm thermal laser to generate a total of three lesions per eye. On Days 2-21, bilateral topical administrations of vehicle, 1% nintedanib, or 1% lenvatinib were performed three times a day. On Day 3, bilateral intravitreal injections of vehicle, 5 μg/eye of rat anti-VEGF antibody, 50 μg/eye of nintedanib, or 50 μg/eye of lenvatinib were performed.

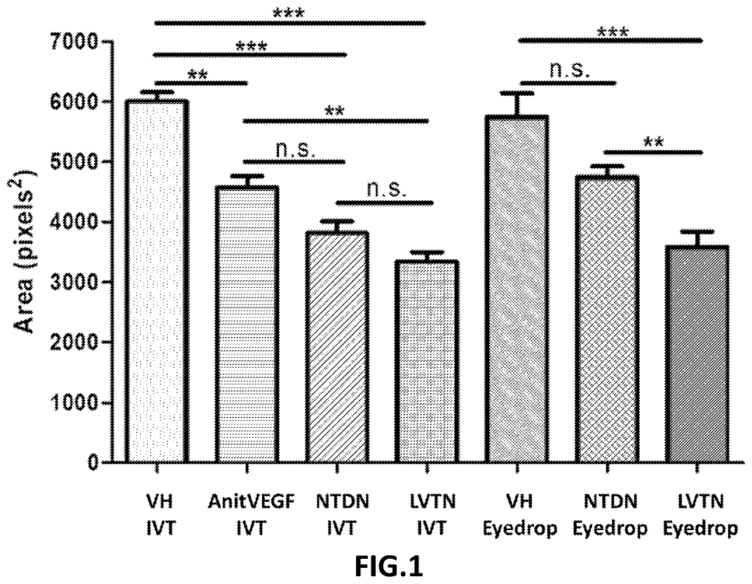

On Day 22, fluorescein angiography was performed on all animals, and the lesion sizes/areas were determined using image analysis software (Image J). As shown in FIG. 1, when delivered via intravitreal injections, nintedanib and lenvatinib treatments significantly reduced lesion sizes, relative to those of the vehicle-treated eyes. In addition, intravitreal injection of lenvatinib also significantly reduced lesion sizes, relative to those of the positive control, rat anti-VEGF.

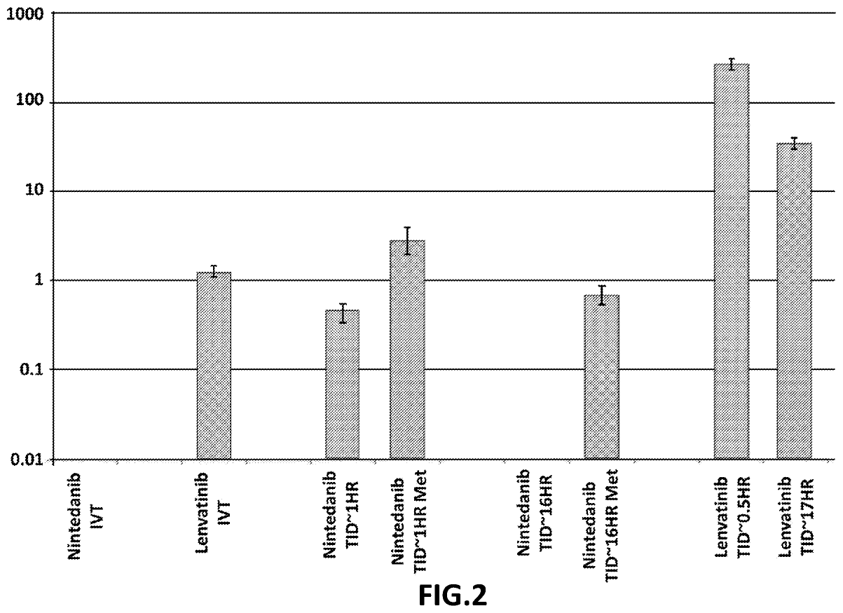

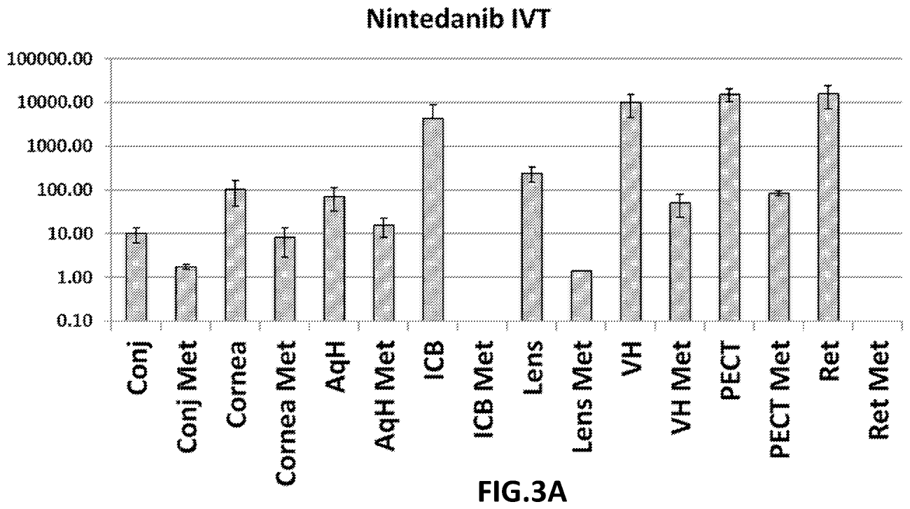

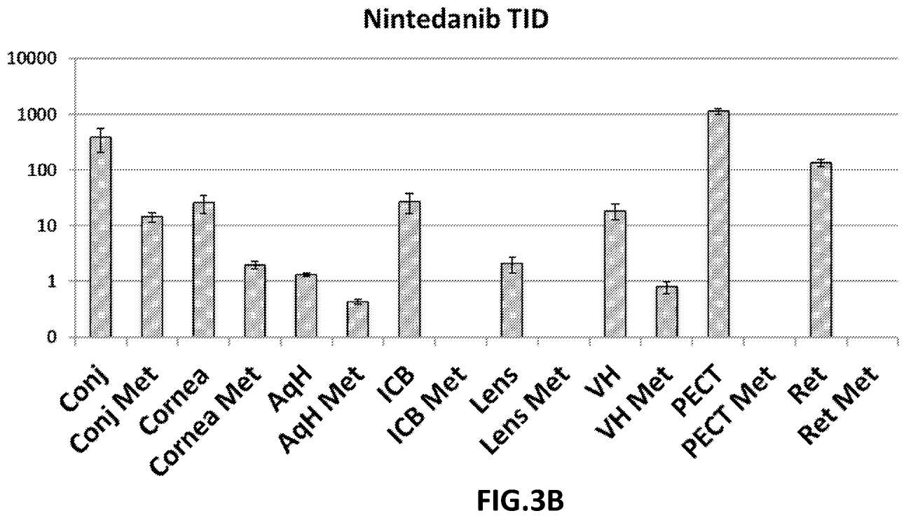

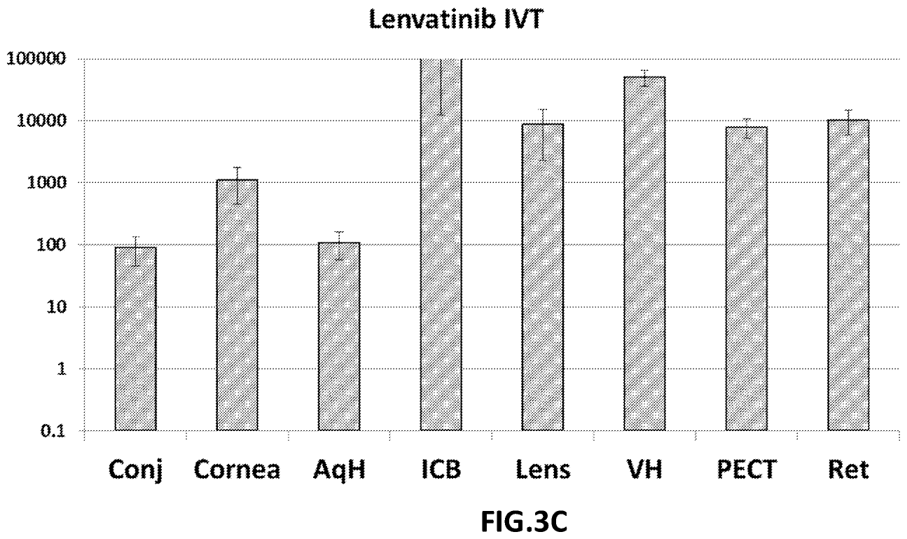

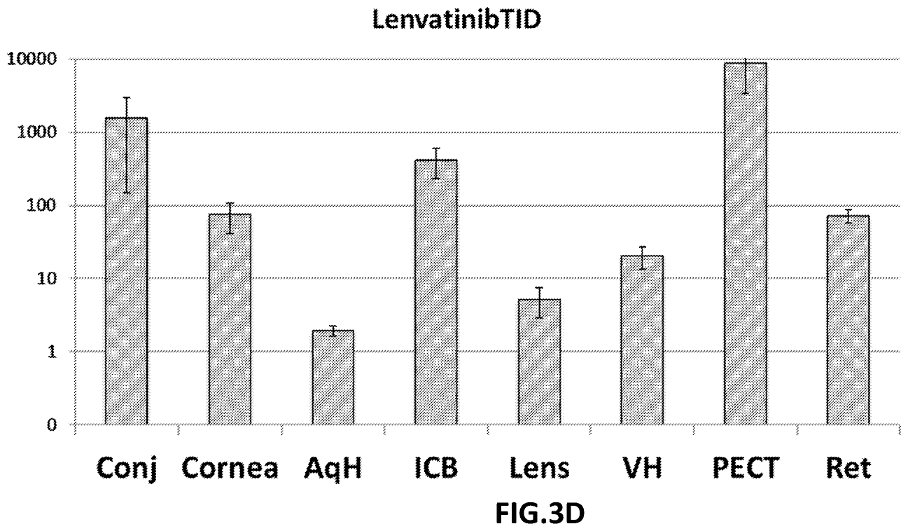

Concentrations of test compounds in ocular tissues and plasma were measured by LCMS-MS after intravitreal or eyedrop administration in rats. Plasma concentrations of nintedanib and its metabolite, and lenvatinib are shown in FIG. 2. Ocular concentrations of nintedanib and its metabolite, and lenvatinib are shown in FIG. 3. These results show that one single intravitreal injection was able to produce tissue concentrations in the back of the eyes comparable to or higher than those from three times daily eye drops over 21 days. The eyedrop administration produced higher drug concentrations in the ocular surface, such as conjunctiva, than the intravitreal injection. Three times daily eyedrop administration maintained higher concentrations in plasma than a single intravitreal injection.

Study results demonstrated that a single intravitreal injection or three times daily ocular instillation of nintedanib and lenvatinib were able to inhibit choroid neovascularization effect for over 20 days and maintained therapeutically effective concentrations in the ocular tissues.

### Example 4

This study evaluated the tolerability and dispersion of the formulations of nintedanib and lenvatinib over 42 days in the posterior segment of the eyes. A single 50 μL intravitreal administration of the 1% suspension was administered to Dutch Belted rabbits. Clinical, Draize, and ophthalmic examination and fundus imaging were conducted regularly during the study. Electroretinography (ERGs) were performed on all animals postdose and immediately prior to the final timepoint.

There were no remarkable formulation-related adverse effects (e.g., ocular irritation, intraocular pressure changes) and no untoward effects in ERGs.

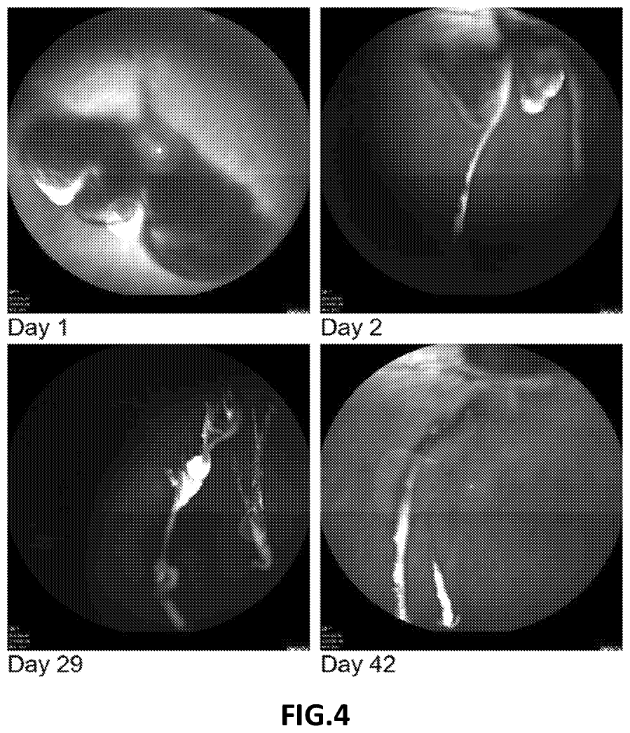

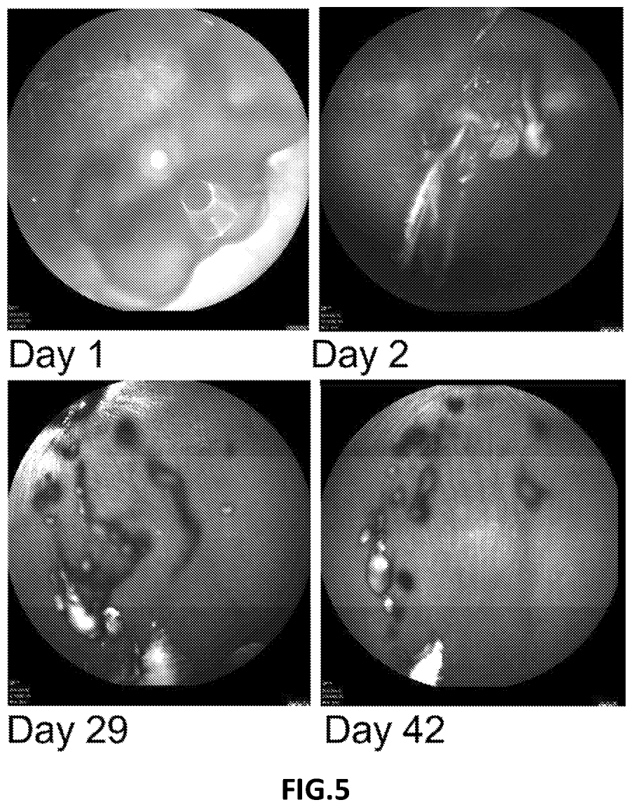

Imaging of the fundus of eyes treated with nintedanib and lenvatinib suspensions indicated that single intravitreal injection resulted in a large globular depot within the mid-vitreous humor within a day of dosing. Within 2 days after injection the bolus was observed to shrink and in some instances appeared as streaks within the mid-vitreous. The depot and streaks continued to decrease in size with time, but remained present out to Day 42 postdose (FIGS. 4 and 5).

For nintedanib, the tissue concentrations in the retina and choroid remained very high on Days 29 and 42 post dosing, higher than 1 μg/gm. Tissues of anterior segments of the eyes (i.e., iris-ciliary body and aqueous humor) had much lower concentrations of nintedanib than the posterior tissues (i.e., vitreous humor, retina, and choroid). Concentrations in conjunctival tissue were mostly under quantitation limit. Approximately 4-7% of the intravitreal dose remained in the vitreous humor on Day 42. The systemic plasma concentrations of nintedanib were relatively steady among days, i.e., 0.252 (0.286) and 0.348 (0.235) ng/mL on Days 29 and 42, respectively.

For lenvatinib, the tissue concentrations in the retina and choroid remained very high on Days 29 and 42 post dosing, approximately over 1 μg/gm. Aqueous humor concentrations had much lower concentrations than those in the posterior tissues (i.e., vitreous humor, retina, and choroid). Approximately 9-17% and 3-9% of the intravitreal dose remained in the vitreous humor on Days 29 and 42. The systemic plasma concentration of lenvatinib were relatively steady among days, i.e., 4.94 and 3.91 ng/mL on Days 29 and 42, respectively.

### Example 5

This study assessed local tolerance and ocular distribution of axitinib, nintedanib, pirfenidone, riociguat, sorafenib and lenvatinib (0.3% w/w) when administered by topical ocular instillation three times daily in the eyes over 10 days. Five to seven male New Zealand White rabbits were used per treatment group. Each eye received a 35 μl eyedrop of vehicle, or respective drugs (0.3%) three times daily for 10 days. After the last dose on Day 11, the animals were sacrificed and eyes were enucleated, and plasma and the ocular tissues were collected. Tissue and plasma concentrations of these compounds were measured by LC-MS/MS.

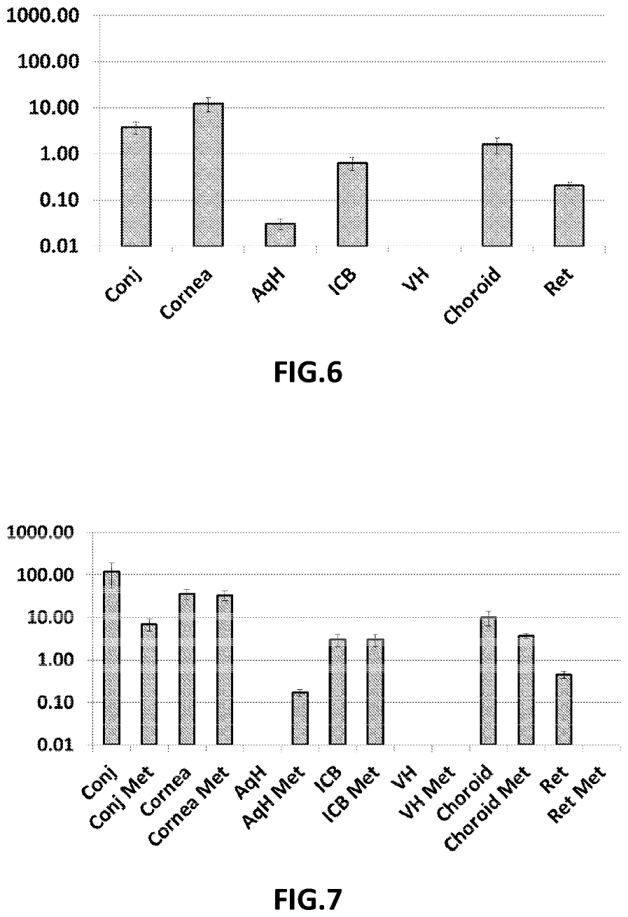

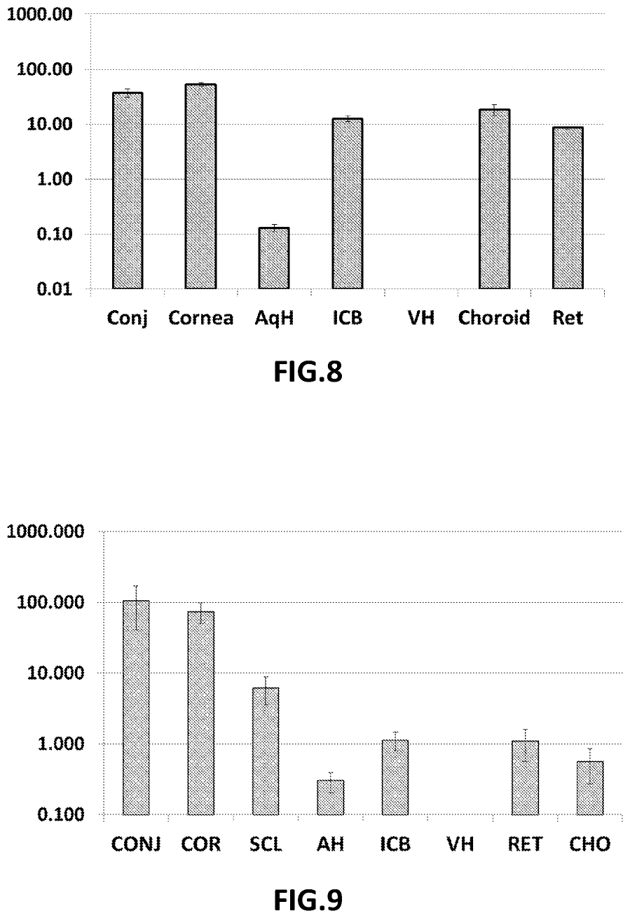

The ocular tissue concentrations of these compounds in various tissues are shown in FIGS. 6-8. Topical instillation of 0.3% w/w of respective drugs delivered high drug concentrations to the anterior tissues of conjunctiva and cornea and significant concentrations in the choroid and retina of rabbits. As this level of drug exposure was maintained in the eyes, very slight to moderate conjunctival congestion and swelling were observed. The extents of these congestion and swelling were similar among the groups (including the vehicle control) over the course of the 10-day observation period.

The plasma concentration, mean (±SD), on Day 11 for axitinib and nintedanib was below the quantifiable limit, for and the metabolite of nintedanib was 1.09 (±0.14) ng/mL, and for lenvatinib was 98.5 (±11) ng/mL.

Study results indicated topical instillation of respective drugs at 0.3% w/w three times daily for 10 days achieved therapeutically effective concentrations in ocular tissues and low plasma concentrations with good topical tolerability and safety in the eyes.

### Example 6

This study evaluated the local tolerance of vehicle, axitinib, pirfenidone, sorafenib and lenvatinib and ocular distribution of axitinib, sorafenib and lenvatinib (0.3% w/w) when administered by topical ocular instillation in the eyes of New Zealand white rabbits (n=6 per group). Each right eye received a 35 μl eyedrop of the vehicle, or respective active drug (0.3% w/w) three times daily for 5 days. On Dosing Day 5, the animals were sacrificed and eyes were enucleated. Ocular and plasma concentrations of select compounds were measured by LC-MS/MS.

Animals among the study groups displayed normal body weight gains over the course of the study. Ocular examinations of the right eye did not show significant findings. Average overall examination scores of all animals in all groups were close to the baseline values for the duration of the study. Intraocular pressure (IOP) was measured using a Tonovet probe. Six consecutive measurements were obtained and the average IOP shown on the display was recorded. IOPs in the right eye remained near to slightly above the baseline values for the duration of the experiment in all groups.

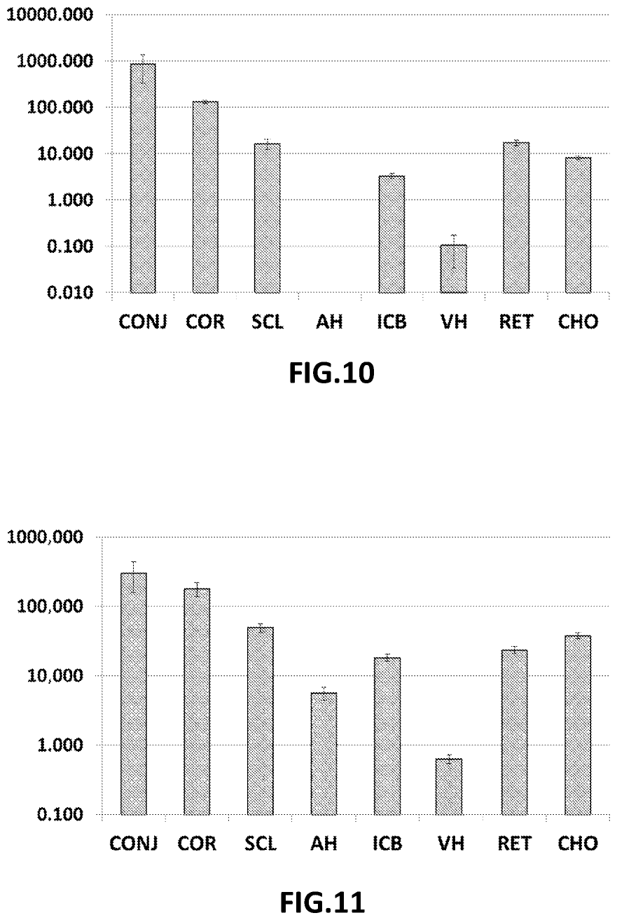

The ocular tissue concentrations for these drugs are listed in FIGS. 9-11. Topical instillation of 0.3% w/w axitinib, sorafenib and lenvatinib delivered high drug concentrations to the anterior tissues of conjunctiva, sclera and cornea and significant concentrations in the choroid and retina of rabbits.

The plasma concentrations, mean (±SD), on Day 5 of dosing were 0.29 (0.15), 5.09 (±1.27) ng/mL and 131 (±24) ng/mL for axitinib, for sorafenib, and lenvatinib.

The above examples show that ophthalmic applications of compounds identified herein can achieve sufficient concentrations to result in therapeutic effects while maintaining good ocular tolerability and safety.

### Example 7

Pharmacokinetics of axitinib and nintedanib was investigated after a single intradermal injection of 0.1% and 1% suspensions to the dorsum of Yucatan minipigs. The dosing volume was 0.1 mL for each injection. Concentrations of drugs in the epidermis and dermis over 28 days at the injection site and at 2 cm away from the injection site were measured using LCMS/MS method.

For both axitinib and nintedanib, there were significant drug concentrations in the skin epidermis and dermis over 28 days post-injection. These concentrations were dose dependent between the 0.1% and 1% doses. The drug concentrations at the injection site were significantly higher than those at 2 cm away, indicating that drug depot was formed upon focal intradermal injection and a prolonged drug diffusion over time from the injection site.

In the skin samples collected at 2 cm away from the injection site, the mean axitinib concentrations ranged from 4.5-55.2 and 42.3-723 ng/gm in the epidermis and 1.2-8 and 2.9-150 ng/gm in the dermis over 28 days after the 0.1% and 1% intradermal injection.

In the skin samples collected at 2 cm away from the injection site, the mean nintedanib concentrations ranged from 2.3-26.3 and 11.6-687 ng/gm in the epidermis and 0.5-5 and 1.8-40.3 ng/gm in the dermis over 28 days after the 0.1% and 1% intradermal injection.

In summary, a single intradermal injection of 0.1% or 1% suspension of axitinib or nintedanib was able to achieve significant drug depot at the site of injection. Each drug would diffuse from this focal depot outward to the adjacent skin area and maintained significantly high and therapeutically meaningful concentrations over a period of 28 days.

### Example 8

This study investigated the pharmacokinetics and distribution of axitinib, sorafenib, pirfenidone, and riociguat after intraprostate injections in Wistar rats (n=6-7 per treatment group). Axitinib, sorafenib and riociguat, as 1% w/w suspension, and pirfenidone as 2.5% suspension were administered as single intraprostate injection (a total of 0.2 mL) on Days 15 and 29 in this 42-day study. Concentrations of test compounds in plasma and prostate were measured using a LCMS/MS method and are shown in Table 5 and Table 6.

TABLE 5
Plasma concentrations (ng/mL) after intraprostate injections in rats.

| Treatment | Study Day | Time | Mean | SEM |
|---|---|---|---|---|
| Axitinib | 15 | 24 h | 1.72 | 0.23 |
| | 29 | 24 h | 1.49 | 0.16 |
| | 42 | 0 h | 0.33 | 0.03 |
| Sorafenib | 15 | 24 h | 84.3 | 8.74 |
| | 29 | 24 h | 83.1 | 10.82 |
| | 42 | 0 h | 20.4 | 2.29 |
| Pirfenidone | 15 | 24 h | 0.381 | 0.09 |
| | 29 | 24 h | 0.454 | 0.13 |
| | 42 | 0 h | BLQ | NA |
| Riociguat | 15 | 24 h | 3.25 | 0.38 |
| | 29 | 24 h | 4.89 | 0.36 |
| | 42 | 0 h | 2.26 | 0.27 |

TABLE 6
Prostate concentrations (μg/μm) after intraprostate injections in rats.

| Treatment | Mean | SEM |
|---|---|---|
| Axitinib | 718 | 287 |
| Sorafenib | 892 | 255 |
| Pirfenidone | 0.014 | 0.004 |
| Riociguat | 492 | 286 |

Study results indicate that there was a prolonged drug release from prostate into systemic circulation after single intraprostate injections for axitinib, riociguat, and sorafenib. The drug concentration in prostate remained high at two weeks after the second biweekly intraprostate injection of axitinib, riociguat and sorafenib.

### Example 9

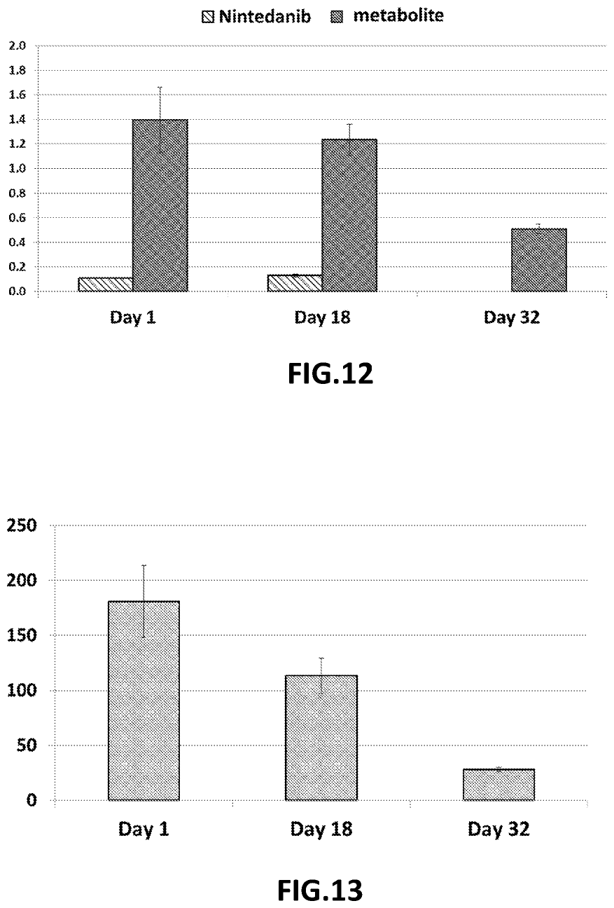

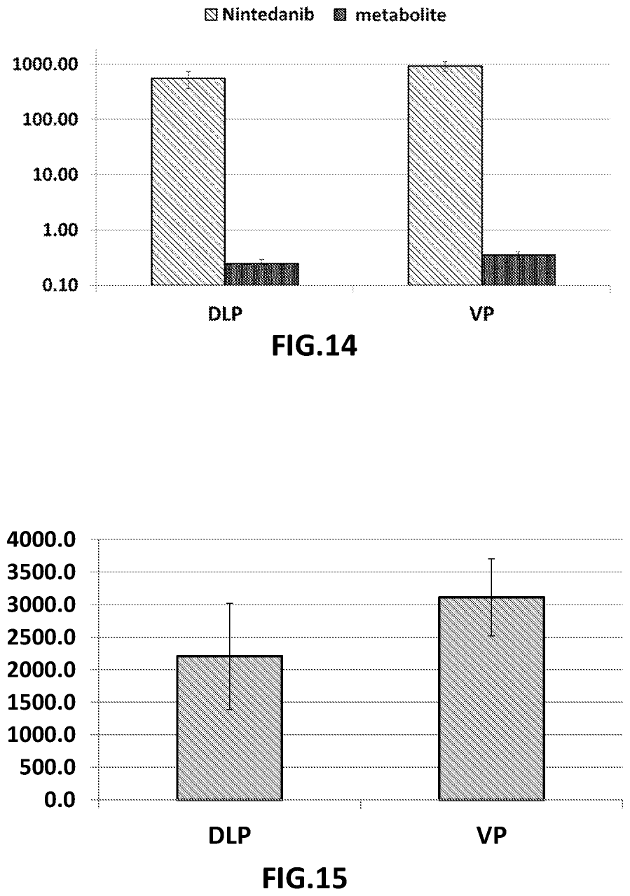

This study investigated the pharmacokinetics and distribution of nintedanib, sunitinib, and lenvatinib after intraprostate injections in Wistar rats (n=5-7 per treatment group). Each compound, as 1% w/w suspension, was administered as single intraprostate injection (a total of 0.4 mL) on Days 1 and 18 in this 32-day study. Concentrations of test compounds in plasma and prostate were measured using a LCMS/MS method and are shown in FIGS. 12-15.

Study results demonstrated significant drug concentrations in the dorsolateral and ventral lobes of prostate. On Day 32, about two weeks after the second intraprostate injection, there were significant amount of test compounds remaining in the prostate, indicating prolonged drug residence. The plasma concentrations of the respective drug were much lower than its prostate concentration, mainly due to the slow diffusion of drug from the prostate depot into the systemic circulation.

### Example 10

The purpose of this study was to evaluate the suspension formulation when administered via intradermal injection with axitinib, nintedanib, sorafenib, and lenvatinib to the dorsal skin along linear incisions of minipigs.

Four male minipigs were dosed. Animals were dosed once via intradermal injection along the edges of each of 10 wound sites for each animal (5 wounds on each side of the dorsum [perpendicular to the spine], approximately 3 cm in length and 3 cm distance from the spine). Axitinib, nintedanib, lenvatinib, and the vehicle control article were administered via eight 0.1 mL intradermal injections around each respective wound site (one injection/cm on both sides of the wound), for a total volume of 0.8 mL/wound site. Sorafenib was administered via the same method at a volume of 0.2 mL/injection, for a total volume of 1.6 mL/wound site. The vehicle control article was Vehicle for Test Articles axitinib, nintedanib, lenvatinib, and sorafenib.

A generalized measure of inflammation, including inflammatory cell infiltrate, fibrin deposition, fibroplasia, epithelial hyperplasia and areas of tissue necrosis were assessed microscopically. The severity of inflammation in control wound sites were slight to moderate over time (Days 4, 7, 9, and 29 post wound). For test agent treated wounds, all were graded as slight on Day 4, and there was no consistent difference over time as compared to the control group.

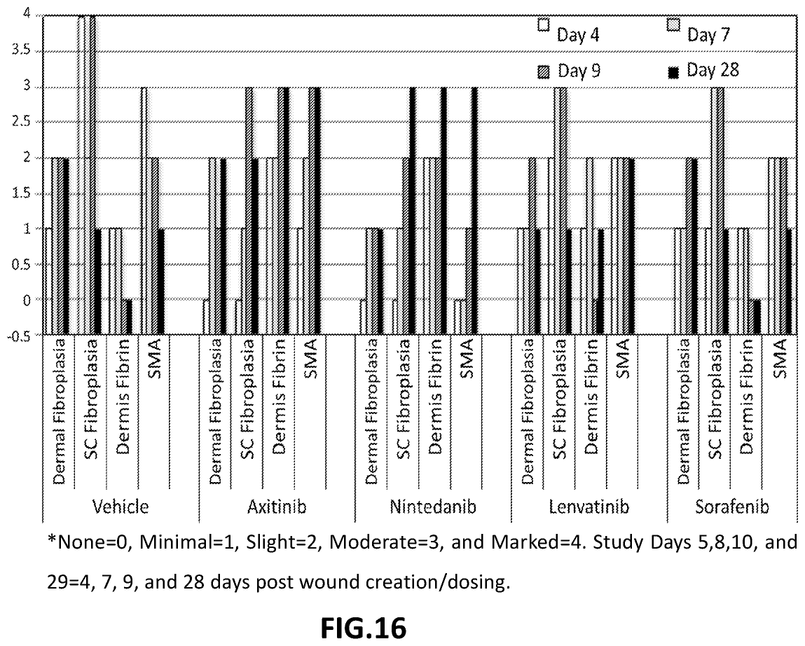

The results are summarized in FIG. 16. Over 29 days, microscopically the vehicle treated wound sites showed minimal to moderate inflammation, moderate to no dermis provisional matrix, minimal to slight dermis fibroplasia, marked to minimal subcutis fibroplasia, slight to marked subcutis mixed cell infiltrate, minimal to none epidermis hyperplasia, minimal to none dermis fibrin, and moderate to minimal SMA staining. Axitinib showed slight to moderate inflammation, marked to moderate dermis provisional matrix, none to slight dermis fibroplasia, none to moderate subcutis fibroplasia, moderate to minimal subcutis mixed cell infiltrate, minimal to slight epidermis hyperplasia, slight to moderate dermis fibrin, and minimal to moderate SMA staining. Nintedanib showed slight to moderate inflammation, marked dermis provisional matrix, none to slight dermis fibroplasia, none to moderate subcutis fibroplasia, slight to marked subcutis mixed cell infiltrate, minimal to moderate epidermis hyperplasia, slight to moderate dermis fibrin, and none to moderate SMA staining. Sorafenib showed moderate to minimal inflammation, slight to none dermis provisional matrix, minimal to slight dermis fibroplasia, minimal to moderate subcutis fibroplasia, slight to marked subcutis mixed cell infiltrate, none epidermis hyperplasia, minimal to none dermis fibrin, and slight to minimal SMA staining. Lenvatinib showed slight to marked inflammation, marked to minimal dermis provisional matrix, minimal to slight dermis fibroplasia, moderate to minimal subcutis fibroplasia, slight to severe subcutis mixed cell infiltrate, slight to none epidermis hyperplasia, minimal to slight dermis fibrin, and slight SMA staining.

In conclusion, these results suggest that axitinib (2% suspension), nintedanib (2% suspension), sorafenib (1% suspension), and lenvatinib (2% suspension) treated wounds demonstrate a delay in granulation tissue formation and a prolonged presence of myofibroblasts compared to the vehicle treated control wounds. The suspension formulation could maintain significantly high and therapeutically meaningful concentrations for a long duration (e.g. at least one month).

While certain embodiments have been illustrated with a limited number of examples, one skilled in the art would appreciate that other modifications and variations are possible without departing from the scope of the disclosure. Therefore, the scope of protection of should not be limited by any particular examples or embodiments.

1. A pharmaceutical composition comprising: an aqueous suspension comprising a pharmaceutically acceptable vehicle and at least one multi-target inhibitor.

2. The pharmaceutical composition of claim 1, wherein the pharmaceutically acceptable vehicle comprises a suspending agent.

3. The pharmaceutical composition of claim 2, wherein the suspending agent is sodium carboxymethylcellulose, cellulose, or a mixture of multiple celluloses.

4. The pharmaceutical composition of claim 1, wherein the pharmaceutically acceptable vehicle comprises a wetting agent.

5. The pharmaceutical composition of claim 1, wherein the pharmaceutically acceptable vehicle comprises a buffer system.

6. The pharmaceutical composition of claim 1, wherein the pharmaceutically acceptable vehicle comprises an osmotic agent.

7. The pharmaceutical composition of claim 1, wherein the multi-target inhibitor is about 0.01% to about 20% of the total weight of the composition.

8. The pharmaceutical composition of claim 7, wherein the multi-target inhibitor is about 0.01% to about 10% of the total weight of the composition.

9. The pharmaceutical composition of claim 8, wherein the multi-target inhibitor is about 0.01% to about 8% of the total weight of the composition.

10. The pharmaceutical composition of claim 1, wherein the composition is injectable.

11. The pharmaceutical composition of claim 1, wherein said multi-target inhibitor comprises axitinib, nintedanib, pirfenidone, riociguat, sorafenib, sunitinib, lenvatinib, regorafenib, ponatinib and pazopanib, or a combination thereof.

12. The pharmaceutical composition of claim 11, wherein said multi-target inhibitor is nintedanib.

13. The pharmaceutical composition of claim 11, wherein said multi-target inhibitor is lenvatinib.

14. The pharmaceutical composition of claim 11, wherein said multi-target inhibitor is axitinib.

15. The pharmaceutical composition of claim 11, wherein said multi-target inhibitor is sunitinib.

16. The pharmaceutical composition of claim 11, wherein said multi-target inhibitor is regorafenib.

17. A method of treating a dermatological disorder, an ophthalmologic disorder, or a urogenitary disorder, comprising: administering an effective amount of the pharmaceutical composition of claim 1, to treat a subject suffering from the dermatological disorder, the ophthalmologic disorder, or the urogenitary disorder.

18. The method of claim 17, wherein the subject suffers from the dermatological disorder, and the dermatological disorder comprises: acne scar, skin scar, wrinkle, cellulite and dermal neoplastic fibrosis, scarring alopecia, vasculopathy, vasculitis, exuberant burn wound healing, diabetic foot syndrome, scleroderma, arthrofibrosis, Peyronie's disease, Dupuytren's contracture, adhesive capsulitis, or a combination thereof.

19. The method of claim 17, wherein the subject suffers from the ophthalmologic disorder, and the ophthalmologic disorder comprises: a choroidal neovascularization associated disease, age-related macular degeneration, compromised corneal transparency, cornea scar formation, pterygia, anterior cataract formation, glaucoma filtration surgery, glaucoma, photorefractive keratectomy, laser in situ keratomileusis, a disorder related to contraction of the pre- and epiretinal membranes, proliferative vitreoretinopathy, proliferative diabetic retinopathy, diabetic macular edema, a disorder related myopic choroidal neovascularization, retinal vain occlusion, sub-retinal fibrosis, sub-retinal scarring, a choroidal membrane related disorder, retinal gliosis, an epiretinal membrane related disorder, glial scar formation, or a combination thereof.

20. The method of claim 17, wherein the subject suffers from the urogenitary disorder, and the urogenitary disorder comprises: benign prostate hyperplasia, a lower urinary tract symptom, benign prostatic enlargement, bladder outlet obstruction, an overactive bladder related disorder, prostatitis, prostatic intraepithelial neoplasia, neurogenic bladder syndrome, prostate cancer, urinary incontinence, prostate cancer, pelvic pain, or a combination thereof.
